(19) United States
(12) Patent Application Publication
(10) Pub. No.: US 2003/0004157 A1
(43) Pub. Date: Jan. 2, 2003

(54) Use of NK-1 receptor antagonists against benign prostatic hyperplasia

(76) Inventors: Susanne Buser, Frenkendorf (CH); Anthony P.D.W. Ford, Mountain View, CA (US); Torsten Hoffmann, Weil am Rhein (DE); Barbara Lenz, Bad Krozingen (DE); Andrew John Sleight, Riedisheim (FR); Pierre Vankan, Basel (CH)

Correspondence Address: Rohan Peries, Roche Bioscience, Patent Law Dept. M/S A2-250, 3401 Hillview Avenue, Palo Alto, CA, 94304, US

(21) Appl. No.: 10/071,570

(22) Filed: Feb. 8, 2002

(30) Foreign Application Priority Data
Apr. 23, 2001 (EP) EP 01109853.0

(51) Int. Cl. A61K 31/55; A61K 31/4439; A61K 31/44; A61K 31/4545

(52) U.S. Cl. 514/217.04; 514/318; 514/346; 514/349; 514/343

(57) ABSTRACT

The invention relates to the use of an NK-1 receptor antagonist for the treatment or prevention of benign prostatic hyperplasia (BPH). The preferred NK-1 receptor antagonists are compounds of the general formula

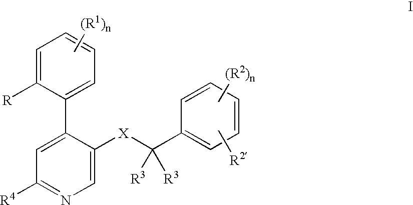

wherein the meanings of R, R¹, R², R^{2′}, R³, R⁴ are explained in the specification and the pharmaceutically acceptable acid addition salts and the prodrugs thereof Preferred compounds are 2-(3,5-bis-trifluoromethyl-phenyl)-N-methyl-N-(6-morpholin-4-yl-4-o-tolyl-pyridin-3-yl)-isobutyramide, 2-(3,5-bis-trifluoromethyl-phenyl)-N-methyl-N-[6-(4-methyl-piperazin-1-yl)-4-o-tolyl-pyridin-3-yl]-isobutyramide, 2-(3,5-bis-trifluoromethyl-phenyl)-N-[6-(1,1-dioxo-1λ⁶-thiomorpholin-4-yl)-4-o-tolyl-pyridin-3-yl]-N-methyl-isobutyramide and 2-(3,5-bis-trifluoromethyl-phenyl)-N-[6-(1,1-dioxo-1λ⁶-thiomorpholin-4-yl)-4-(4-fluoro-2-methyl-phenyl)-pyridin-3-yl]-N-methyl-isobutyramide. The invention also relates to pharmaceutical composition comprising one or more such NK-1 receptor antagonists and a pharmaceutically acceptable excipient for the treatment and/or prevention of benign prostatic hyperplasia.

This application claims benefit under Title 35, U.S. Code, § 119 of European Patent Application No. 01109853.0, filed on Apr. 23, 2001.

### BACKGROUND OF THE INVENTION

The present invention concerns NK-1 receptor antagonists and their use for the treatment and/or prevention of benign prostatic hyperplasia (BPH).

Benign prostatic hyperplasia (BPH) is quite common in older men. Its symptoms may interfere with daily activities and impact the perception of wellbeing and thus the quality of life. BPH can be progressive and lead to urinary retention, infections, bladder calculi and renal failure. While moderate symptoms may remain untreated, bothersome symptoms and complications may need medical therapy or surgery.

Catheterization may be needed in case of an acute urinary retention, one of the complications caused by BPH. There are two different forms of acute urinary retention, viz. spontaneous or precipitated acute urinary retention, whereby the first one is often considered by patients to be the most serious outcome of BPH. Spontaneous acute urinary retention can be treated with 5-alpha-reductase inhibitors, such as finasteride as described by Andersen et al., Urology, 49(6), 839-845, (1997). Precipitated acute urinary retention is an episode of acute urinary retention which often occurs within the first three days after anesthesia or surgery, after a stroke or a congestive heart failure; a medical condition such as prostatitis or urinary tract infection; or ingestion of medication or drugs known to precipitate retention, e.g., pseudoephedrine hydrochloride, cold medicine, pain medication such as narcotics or sedatives, or benadryl.

Benign prostatic hyperplasia (BPH) is unusual in that it occurs spontaneously as a clinical disease in males of only two species, humans and dogs (Emberton M. and Mundy A. R. (1999), “The Prostate and Benign Prostatic Hyperplasia”, in The Scientific Basis of Urology, editors Mundy, Fitzpatrick, Neal & George; Isis Medical Media, Oxford UK. 257pp.). Anatomical similarities between canine and human prostate were first extensively reviewed by Price D. in “Comparative aspects of development and structure in the prostate”. Natl. Cancer Inst. Monogr., 12, 1-7, (1963), through developmental studies: the canine prostate surrounds the neck of the bladder and proximal urethra, grossly resembling the human prostate, is of mixed stromal and glandular morphology and is ensheathed in a capsule of smooth muscle, fibrovascular tissue, nerves and ganglia. In BPH of dogs and in men, the epithelial and stromal prostatic elements both increase in amount in a seemingly uncoordinated fashion (see Strandberg J. D. in “Comparative Pathology of Benign Prostatic Hyperplasia”, in, Prostatic Diseases, editor Lepor H., W. B. Saunders Company, Philadelphia (2000)). Accordingly, dogs have been used extensively in experimental studies of the etiology, pathogenesis and treatment of BPH (Walsh P. C. and Wilson J. D., “The induction of prostatic hypertrophy in the dog with androstanediol”, J. Clin. Invest., 57, 1093-7, (1976); Suzuki K., Okazaki H., Ono Y., Kurokawa K., Suzuki T., Onuma E., Takanashi H., Mamiya Y. and Yamanaka H., “Effect of dual inhibition of 5-a-reductase and aromatase on spontaneously developed canine prostatic hypertrophy”, Prostate (NY), 37(2), 70-76, (1998); for a review see also Strandberg J. D. (2000; cited above).

Neurokinin-1 (NK-1) or substance P is a naturally occurring undecapeptide belonging to the tachykinin family of peptides, the latter being so-named because of their prompt contractile action on extravascular smooth muscle tissue. The receptor for neurokinin-1 or substance P is a member of the superfamily of G protein-coupled receptors and is named NK-1 receptor. This receptor is widely distributed throughout the mammalian nervous system (especially brain and spinal ganglia) and is also present in the circulatory system and in peripheral tissues (especially the duodenum, the jejunum and the genito-urinary tract). The receptor is believed to be involved in the regulation of a number of diverse biological processes as outlined below.

The central and peripheral actions of the mammalian tachykinin substance P have been associated with numerous inflammatory conditions including migraine, rheumatoid arthritis, asthma, and inflammatory bowel disease as well as mediation of the emetic reflex and the modulation of central nervous system (CNS) disorders such as Parkinson's disease (Neurosci. Res., 7, 187-214, (1996)), anxiety (Can. J. Phys., 75, 612-621, (1997)) and depression (Science, 281, 1640-1645, (1998)).

Evidence for the usefulness of tachykinin receptor antagonists in pain, headache, especially migraine, Alzheimer's disease, multiple sclerosis, attenuation of morphine withdrawal, cardiovascular changes, edema, such as edema caused by thermal injury, chronic inflammatory diseases such as rheumatoid arthritis, asthma/bronchial hyperreactivity and other respiratory diseases including allergic rhinitis, inflammatory diseases of the gut including ulcerative colitis and Crohn's disease, ocular injury and ocular inflammatory diseases reviewed in “Tachykinin Receptor and Tachykinin Receptor Antagonists”, J. Auton. Pharmacol., 13, 23-93, (1993).

Furthermore, neurokinin-1 receptor antagonists are being developed for the treatment of a number of physiological disorders associated with an excess or imbalance of tachykinin, in particular substance P. Examples of conditions in which substance P has been implicated include disorders of the central nervous system such as anxiety, depression and psychosis (International Patent Application, Publication Nos. WO 95/16679, WO 95/18124 and WO 95/23798).

The neurokinin-1 receptor antagonists are further believed to be useful for the treatment of motion sickness and for treatment induced vomiting.

The reduction of cisplatin-induced emesis by a selective neurokinin-1-receptor antagonist is described in The New England Journal of Medicine, Vol. 340, No. 3, 190-195, (1999).

Furthermore, U.S. Pat. No. 5,972,938 describes a method for treating a psychoimmunologic or a psychosomatic disorder by administration of a tachykinin receptor, such as the NK-1 receptor antagonist.

The usefulness of neurokinin 1 receptor antagonists for the treatment of certain forms of urinary incontinence is furthermore described in Neuropeptides, 32(1), 1-49, (1998) and Eur. J. Pharmacol., 383(3), 297-303, (1999).

NK-1 receptor antagonists have been reported to have also a beneficial effect in the therapy of traumatic brain injury (oral disclosure by Prof. Nimmo at the International Tachykinin Conference 2000 in La Grande Motte, France, Oct. 17-20, 2000 with the title “Neurokinin 1 (NK-1) Receptor Antagonists Improve the Neurological Outcome Following Traumatic Brain Injury”, Authors: Nimmo A. J., Bennett C. J., Hu X., Cernak I., Vink R.).

The use of NK-1 receptor antagonists for the treatment or prevention of chronic nonbacterial prostatitis and prostatodynia has been described in International Patent Publication No. WO 99/59583.

International Patent Publication No. WO 01/01922 describes the use of substance P antagonists in the treatment of the adenocarcinomas, particularly genito-urinary tract neoplasms such as prostatic carcinoma.

### SUMMARY OF THE INVENTION

It has now been found that surprisingly antagonists of the neurokinin 1 (NK-1, substance P) receptor can be used in the treatment and/or prevention of benign prostatic hyperplasia.

The present invention therefore relates to the use of an NK-1 receptor antagonist for the treatment or prevention of benign prostatic hyperplasia.

The present invention also relates to the use of an NK-1 receptor antagonist for the manufacture of a medicament for the treatment and/or prevention of benign prostatic hyperplasia.

The invention also relates to a method of treating or preventing benign prostatic hyperplasia in a mammal, including a human, by administering an effective amount of an NK-1 receptor antagonist.

The invention also relates to a pharmaceutical composition comprising one or more NK-1 receptor antagonists and a pharmaceutically acceptable excipient for the treatment and/or prevention of benign prostatic hyperplasia. Said NK-1 receptor antagonist may be present in the form of a pharmaceutically acceptable acid addition salt or may be present in the form of a prodrug, preferably in the form of an N-oxide.

### DETAILED DESCRIPTION OF THE INVENTION

The terms “NK-1 receptor antagonist” and “Substance P receptor antagonist” are used herein refer to any synthetic chemical compound that inhibits binding of substance P to the NK-1 receptor. Preferably the pKi of the NK-1 receptor antagonists for the NK-1 receptor will be greater than 7 (i.e. an affinity of 100nM), more preferably in the range of 8-10 (i.e. an affinity of 10nM to 0.1nM). Binding affinities may be determined by the method described in the Examples. A large number of such receptor antagonists are known and have been described e.g. in European Patent Publication No. EP-A-1,035,115 of Boes M., Branca Q., Galley G., Godel T., Hoffmann T., Hunkeler W., Schnider P., Stadler H., entitled “Preparation of N-benzyl-4-tolylnicotinamides and related compounds as neurokinin-1 receptor antagonists.”. This document as well as all documents referred to below are herewith incorporated by reference in their entirety. It has now been found that the selective NK-1 receptor antagonist of the general formula

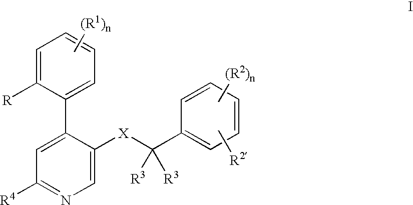

wherein

R is hydrogen, lower alkyl, lower alkoxy, halogen or trifluoromethyl;

R¹ is hydrogen or halogen; or

R and R¹ may be together -CH═CH-CH═CH-;

R² and R²′ are independently from each other hydrogen, halogen, trifluoromethyl, lower alkyl, lower alkoxy or cyano; or

R² and R²′ may be together -CH═CH-CH═CH-, optionally substituted by one or two substituents selected from lower alkyl, halogen or lower alkoxy;

R³ is, independently from each other if occurring twice, hydrogen, lower alkyl or may, if occurring twice, form together with the carbon atom to which they are attached a cycloalkyl group;

R⁴ is hydrogen, -N(R⁵)₂, -N(R⁵)(CH₂)ₙ,OH, -N(R⁵)S(O)₂-lower alkyl, -N(R⁵)S(O)₂-phenyl, -N═CH-N(R⁵)₂, -N(R⁵)C(O)R⁵, a cyclic tertiary amine of the group

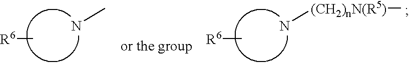

or R⁴ is -(C≡C)ₙR⁷ or -(CR′═CR″)ₙR⁷

wherein R⁷ is

a) halogen,

b) cyano, or the following groups:

c) -(CR′R″)ₙ-R⁸,

d) -C(O)NR′R″,

e) -C(O)O(CH₂)ₙR⁸,

f)-C(O)R⁸,

g) -N(OH)-(CH₂)ₙR⁸,

h) -NR′C(O)-(CH₂)ₙR⁸,

i) -N[C(O)-R′]₂,

j) -OR⁹,

k) -(CH₂)ₙ-SR⁹, -(CH₂)ₙ-S(O)R⁹, or -(CH₂)ₙ-S(O)₂R⁹,

l) aryl, optionally substituted by one or more substituents, selected from halogen, trifluoromethyl, lower alkyl, lower alkoxy, cyano, hydroxy, -NR′R″, nitro, -(CH₂)ₘOR′, -C(O)NR′R″, -C(O)OR′ or -C(O)R′,

m) is a five or six membered heteroaryl group, containing one to four heteroatoms, selected from N, O or S and may be optionally substituted by one or more substituents, selected from halogen, trifluoromethyl, lower alkyl, lower alkoxy, cyano, hydroxy, -NR′R″, nitro, -(CH₂)ₘOR′, -C(O)OR′, -C(O)NR′R″ or -C(O)R′,

n) is a five or six membered saturated cyclic tertiary amine of the group

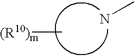

which may contain one additional heteroatom, selected from N, 0 or S,

R′/R″ are independently from each other hydrogen, hydroxy, lower alkyl, cycloalkyl or aryl, wherein the lower alkyl, cycloalkyl or aryl group may be optionally substituted by one or more substituents, selected from halogen, trifluoromethyl, lower alkyl, lower alkoxy, cyano, hydroxy, -NR′″R″″, nitro, -(CH₂)ₘOR′″, -C(O)NR′″R″″, -C(O)OR′″ or-C(O)R′″,

R′″/R″″ are independently from each other hydrogen, lower alkyl, cycloalkyl or aryl,

R⁸ is hydrogen, cyano, hydroxy, halogen, trifluoromethyl, -C(O)OR′, -OC(O)R or aryl, optionally substituted by one or more substituents, selected from halogen, trifluoromethyl, lower alkyl, lower alkoxy, cyano, hydroxy, -NR′R″ nitro, -(CH₂)ₘOR′, -C(O)NR′R″, -C(O)OR′ or -C(O)R′, or is a five or six membered heteroaryl group, containing one to four heteroatoms, selected from N, O or S and may be optionally substituted by one or more substituents, selected from halogen, trifluoromethyl, lower alkyl, lower alkoxy, cyano, hydroxy, -NR′R″, nitro, -(CH₂)ₘOR′, -C(O)NR′R″, -C(O)OR′ or -C(O)R′,

R⁹ is hydrogen, lower alkyl, trifluoromethyl, or aryl, wherein the lower alkyl or aryl group may be optionally substituted by one or more substituents, selected from halogen, trifluoromethyl, lower alkyl, lower alkoxy, cyano, hydroxy, -NR′R″, nitro, -C(O)NR′R″, -(CH₂)ₘOR′, -C(O)OR′ or -C(O)R′, or is a five or six membered heteroaryl group, containing one to four heteroatoms, selected from N, O or S and may be optionally substituted by one or more substituents, selected from halogen, trifluoromethyl, lower alkyl, lower alkoxy, cyano, hydroxy, -NR′R″, nitro, -(CH₂)ₘOR′, -C(O)NR′R″, -C(O)OR′ or -C(O)R′,

R¹⁰ is -C(O)-(CH₂)ₙOH or an oxo group; or R⁴is an N-oxide of the general formula

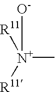

wherein R¹¹ and R^{11′}are independently from each other -(CH₂)ₚOR¹² or lower alkyl,

wherein R¹² is hydrogen, lower alkyl or phenyl; or

R¹¹ and R^{11′}form together with the N-atom to which they are attached a cyclic tertiary amine of the group

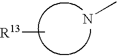

wherein R¹³ is hydrogen, hydroxy, lower alkyl, lower alkoxy, -(CH₂)ₚOH, -COOR³, -CON(R³)₂, -N(R³)CO-lower alkyl or -C(O)R³;

R⁵ is, independently from each other, hydro gen, C₃₋₆-cycloalkyl, benzyl, phenyl or lower alkyl;

R⁶ is hydrogen, hydroxy, lower alkyl, -(CH₂)ₙCOO-lower alkyl, -N(R⁵)CO-lower alkyl, hydroxy-lower alkyl, cyano, -(CH₂)ₙO(CH₂)ₙOH, -CHO or a 5-or 6 membered heterocyclic group, optionally bonded via an alkylene group;

X is -C(O)N(R⁵)-, -(CH₂)ₚO-, -O(CH₂)ₚ-, -(CH₂)ₚN(R⁵)-, -N(R⁵)C(O)-, or -N(R⁵)(CH₂)ₚ-;

n is 0, 1, 2, 3 or 4;

m is 1 or 2; and

p is 1, 2,or3;

and the pharmaceutically acceptable acid addition salts and the prodrugs thereof are particularly suitable for the treatment and/or prevention of benign prostatic hyperplasia.

The present invention also relates to the use of an NK-1 receptor antagonist of the general formula (I) for the manufacture of a medicament for the treatment and/or prevention of benign prostatic hyperplasia.

The invention also relates to a method of treatment and/or prevention of benign prostatic hyperplasia in a mammal, including a human, by administering an effective amount of an NK-1 receptor antagonist of the general formula (I) and a pharmaceutically acceptable excipient.

The invention also relates to a pharmaceutical composition comprising one or more NK-1 receptor antagonists and a pharmaceutically acceptable excipient for the treatment and/or prevention of benign prostatic hyperplasia. Said NK-1 receptor antagonist may be present in the form of a pharmaceutically acceptable acid addition salt or may be present in the form of a prodrug, preferably in the form of an N-oxide.

This patent application also describes preferred NK-1 receptor antagonists of the present invention, viz. compounds of the general formula (I) wherein R¹ and R^{1′}and R³ have the meaning specified above and

R² and R^{2′}are independently from each other hydrogen, halogen, trifluoromethyl, lower alkoxy or cyano; or

R² and R^{2′}may be together -CH═CH-CH═CH-, optionally substituted by one or two substituents selected from lower alkyl or lower alkoxy;

R⁴ is hydrogen, -N(R⁵)₂, -N(R⁵)(CH₂)ₙOH, -N(R⁵)S(O)₂-lower alkyl, -N(R⁵)S(O)₂-phenyl, -N═CH-N(R⁵)₂, -N(R⁵)C(O)R⁵ or a cyclic tertiary amine of the group or the group

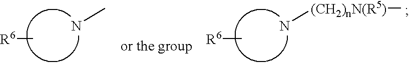

R⁵ is, independently from each other, hydrogen, C₃-₆-cycloalkyl, benzyl or lower alkyl;

R⁶ is hydrogen, hydroxy, lower alkyl, -(CH₂)ₙCOO-lower alkyl, -N(R⁵)CO-lower alkyl, hydroxy-lower alkyl, cyano, -(CH₂)ₙO(CH₂)ₙOH, -CHO or a 5-or 6 membered heterocyclic group, optionally bonded via an alkylene group;

X is -C(O)N(R⁵)-, -(CH₂)ₘO-, -(CH₂)ₘN(R⁵)-, -N(R⁵)C(O)-, or -N(R⁵)(CH₂)ₘ-;

n is 0, 1, 2, 3 or 4; and

m is 1 or 2;

and the pharmaceutically acceptable acid addition salts and the prodrugs thereof

The following definitions of the general terms used in the present description apply irrespective of whether the terms in question appear alone or in combination.

As used herein, the term “lower alkyl” denotes a saturated straight- or branched-chain alkyl group containing from 1 to 7 carbon atoms, for example, methyl, ethyl, propyl, isopropyl, n-butyl, i-butyl, 2-butyl, t-butyl and the like. Preferred lower alkyl groups are groups with 1 to 4 carbon atoms.

The term “lower alkoxy” denotes a group wherein the alkyl residues are as defined above and which is attached via an oxygen atom.

The term “halogen” denotes chlorine, iodine, fluorine and bromine.

The term “cycloalkyl” denotes a saturated carbocyclic group containing 3 to 6 carbon atoms.

The term “cyclic tertiary amine” denotes, for example, pyrrolidin-1-yl, imidazol-1-yl, piperidin-1-yl, piperazin-1-yl, morpholin-4-yl, thiomorpholin-4-yl, 1-oxo-thiomorpholin-4-yl, 1,1-dioxo-thiomorpholin-4-yl, 2,3-dihydro-[1,4] oxazin-4-yl, or [1,2,4]triazol-1-yl.

The term “five or six membered heteroaryl group, containing one to four heteroatoms, selected from N, O or S” denotes, for example, the following groups: pyrrol-1-yl, imidazol-1 or 2-yl, pyrazol-1-yl, pyridin-2, 3 or 4-yl, pyrazinyl, pyrimidinyl, pyridazinyl, isothiazolyl, isoxazolyl, thienyl, 1,2,3-triazolyl, 1,2,4-oxadiazolyl, tetrahydro-pyridinyl, isoxazolyl or furyl.

The term “five or six membered saturated cyclic tertiary amine” denotes, for example, pyrrolidinyl, imidazolidinyl, pyrazolidinyl, piperidinyl, piperazinyl, morpholinyl, thiomorpholinyl, thiomorpholin-1,1-dioxo or thiomorpholin-1-oxo.

The term “5 or 6 membered heterocyclic group” denotes, for example pyridinyl, pyrimidinyl, oxadiazolyl, triazolyl, tetrazolyl, thiazolyl, thienyl, furyl, pyranyl, pyrrolyl, imidazolyl, pyrazolyl, isothiazolyl, piperazinyl or piperidyl.

The term “aryl” denotes a monocyclic aromatic hydrocarbon radical or a bicyclic or tricyclic ring system in which at least one ring is aromatic, preferred are phenyl, benzyl or naphthyl rings.

The term “pharmaceutically acceptable acid addition salts” embraces salts with inorganic and organic acids, such as hydrochloric acid, nitric acid, sulfuric acid, phosphoric acid, citric acid, formic acid, fumaric acid, maleic acid, acetic acid, succinic acid, tartaric acid, methanesulfonic acid, p-toluenesulfonic acid and the like. “Treating” or “treatment” of a disease includes:

(1) preventing the disease, i.e. causing the clinical symptoms of the disease not to develop in a mammal that may be exposed to or predisposed to the disease but does not yet experience or display symptoms of the disease,

(2) inhibiting the disease, i.e., arresting or reducing the development of the disease or its clinical symptoms, or

(3) relieving the disease, i.e., causing regression of the disease or its clinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

Preferred compounds for the claimed use are the exemplary compounds in which X in general formula (I) is -C(O)N(R⁵)- and wherein R⁵ is methyl, ethyl or cyclopropyl, for example the following compounds:

N-(3,5-bis-trifluoromethyl-benzyl)-N-methyl-4-o-tolyl-nicotinamide,

N-(3,5-bis-trifluoromethyl-benzyl)-N-methyl-4-(2-chloro-phenyl)-nicotinamide,

N-(3,5-bis-trifluoromethyl-benzyl)-N-methyl-4-(2-trifluoromethyl-phenyl)-nicotinamide,

N-(3,5-bis-trifluoromethyl-benzyl)-N-methyl-4-(2-fluoro-phenyl)-nicotinamide,

N-(3,5-bis-trifluoromethyl-benzyl) -N-methyl-4-(2-methoxy-phenyl) -nicotinamide,

N-(3,5-bis-trifluoromethyl-benzyl)-N-methyl-4-phenyl-nicotinamide,

N-(3,5-bis-trifluoromethyl-benzyl)-N-ethyl-4-o-tolyl-nicotinamide,

N-(3,5-bis-trifluoromethyl-benzyl)-N-cyclopropyl-4-o-tolyl-nicotinamide,

N-[1-(3,5-bis-trifluoromethyl-phenyl)-ethyl]-N-methyl-4-o-tolyl-nicotinamide,

N-(3,5-di-fluorobenzyl)-N-methyl-4-o-tolyl-nicotinamide,

N-(3,5-di-chlorobenzyl)-N-methyl-4-o-tolyl-nicotinamide,

N-(3,5-bis-trifluoromethyl-benzyl)-N-methyl-6-(4-methyl-piperazin-1-yl)-4-o-tolyl-nicotinamide,

2′-methyl-5-(4-methyl-piperazin-1-yl)-biphenyl-2-carboxylic acid-(3,5-bis-trifluoromethyl-benzyl) -methyl-amide,

N-(3,5-bis-trifluoromethyl-benzyl)-N-methyl-6-(4-methyl-piperazin-1-yl)-4-naphthalen-1-yl-nicotinamide,

(4-{5- [(3,5-bis-trifluoromethyl-benzyl)-methyl-carbamoyl]-4-o-tolyl-pyridin-2-yl}-piperazin-1-yl)-acetic acid ethyl ester,

5′-[(3,5-bis-trifluoromethyl-benzyl)-methyl-carbamoyl]-4′-o-tolyl-3,4,5,6-tetrahydro-2H- [1,2′]bipyridinyl-4-carboxylic acid ethyl ester,

N-(3,5-bis-trifluoromethyl-benzyl)-N-methyl-6-(4-propyl-piperazin-1-yl)-4-o-tolyl-nicotinamide,

(RS)-6-[3-(acetyl-methyl-amino)-pyrrolidin-1-yl]-N-(3,5-bis-trifluoromethyl-benzyl) -N-methyl-4-o-tolyl-nicotinamide,

N-(3,5-bis-trifluoromethyl-benzyl) -N-methyl-6-[methyl- (2-morpholin-4-yl-ethyl)-amino]-4-o-tolyl-nicotinamide,

N-( 3,5-bis-trifluoromethyl-benzyl)-N-methyl-6-morpholin-4-yl-4-o-tolyl-nicotinamide,

N-(3,5-bis-trifluoromethyl-benzyl)-N-methyl-6-thiomorpholin-4-yl-4-o-tolyl-nicotinamide,

N-(3,5-bis-trifluoromethyl-benzyl)-N-methyl-6-(1-oxo-14-thiomorpholin-4-yl)-4-o-tolyl-nicotinamide,

N-(3,5-bis-trifluoromethyl-benzyl)-6-(1,1-dioxo-1X⁶-thiomorpholin-4-yl)-N-methyl-4-o-tolyl-nicotinamide,

N-(3,5-bis-trifluoromethyl-benzyl) -N-methyl-6-piperazin-1-yl-4-o-tolyl-nicotinamide,

N-(3,5-bis-trifluoromethyl-benzyl)-6- [4-(2-hydroxy-ethyl)-piperazin-1-yl]-N-methyl-4-o-tolyl-nicotinamide,

N-(3,5-bis-trifluoromethyl-benzyl)-6-(4-cyanomethyl-piperazin-1-yl)-N-methyl-4-o-tolyl-nicotinamide,

N-(3,5-bis-trifluoromethyl-benzyl) -6-{4-[2- (2-hydroxy-ethoxy) -ethyl]-piperazin-1-yl}-N-methyl-4-o-tolyl-nicotinamide,

N-(3,5-bis-trifluoromethyl-benzyl)-N-methyl-6-(4-[1,2,4] oxadiazol-3- yl-methyl-piperazin-1-yl)-4-o-tolyl-nicotinamide,

N-(3,5-bis-trifluoromethyl-benzyl) -N-methyl-6-[4-(5-oxo-4,5-dihydro-1H-[1,2,4]triazol-3-yl-methyl)-piperazin-1-yl]-4-o-tolyl-nicotinamide,

N-(3,5-bis-trifluoromethyl-benzyl)-6-(4-formyl-piperazin-1-yl)-N-methyl-4-o-tolyl-nicotinamide, and

N-methyl-N-(2-methyl-naphthalen-1-yl-methyl)-6-morpholin-4-yl-4-o-tolyl-nicotinamide.

Further preferred compounds for the claimed use are the exemplary compounds in which X in general formula (I) is -N(R⁵)-CO- and wherein R⁵ is hydrogen or methyl, for example the following compounds:

2-(3,5-bis-trifluoromethyl-phenyl)-N-methyl-N-[6-(4-methyl-piperazin-1-yl)-4-o-tolyl-pyridin-3-yl]-isobutyramide,

2-(3,5-bis-trifluoromethyl-phenyl)-N-[4-(2-chloro-phenyl)-6-(4-methyl-piperazin-1-yl)-pyridin-3-yl]-N-methyl-isobutyramide,

2-(3,5-bis-trifluoromethyl-phenyl)-N-[4-(4-fluoro-2-methyl-phenyl)-6-(4-methyl-piperazin-1-yl) -pyridin-3-yl]-N-methyl-isobutyramide,

2-(3,5-bis-trifluoromethyl-phenyl)-N-[4-(2-chloro-phenyl)-pyridin-3-yl]-N-methyl-isobutyramide,

2-(3,5-bis-trifluoromethyl-phenyl-N-methyl-N-(4-o-tolyl-pyridin-3-yl)-isobutyramide,

2-(3,5-bis-trifluoromethyl-phenyl)-N-(4-o-tolyl-pyridin-3-yl)-isobutyramide,

2-(3,5-bis-trifluoromethyl-phenyl)-N-methyl-N-(4-o-tolyl-pyridin-3-yl)-acetamide,

2-(3,5-bis-trifluoromethyl-phenyl)-N-methyl-N-(4-o-tolyl-pyridin-3-yl)-propionamide,

2-(3,5-bis-trifluoromethyl-phenyl)-N-methyl-N-(6-morpholin-4-yl-4-o-tolyl-pyridin-3-yl)-isobutyramide,

2-(3,5-bis-trifluoromethyl-phenyl)-N-[4-(2-chloro-phenyl)-6-morpholin-4-yl-pyridin-3-yl]-N-methyl-isobutyramide,

2-(3,5-bis-trifluoromethyl-phenyl)-N-methyl-N-{6- [methyl-(2-morpholin-4-yl-ethyl)-amino]-4-o-tolyl-pyridin-3-yl}-isobutyramide,

2-(3,5-bis-trifluoromethyl-phenyl)-N-methyl-N-[6-(4-pyrimidin-2-yl-piperazin-1-yl)-4-o-tolyl-pyridin-3-yl]-isobutyramide,

2-(3,5-bis-trifluoromethyl-phenyl)-N-(6-morpholin-4-yl-4-o-tolyl-pyridin-3-yl)-isobutyramide,

2-(3,5-bis-trifluoromethyl-phenyl)-N-[4-(2-chloro-phenyl)-6-dimethylamino-pyridin-3-yl]-isobutyramide,

2-(3,5-bis-trifluoromethyl-phenyl)-N-methyl-N-(6-piperazin-1-yl-4-o-tolyl-pyridin-3-yl)-isobutyramide,

2-(3,5-bis-trifluoromethyl-phenyl)-N-(4-hydroxy-4′-o-tolyl-3,4,5,6-tetrahydro-2H-[1,2′]bipyridinyl-5′-yl)-N-methyl-isobutyramide,

2- (3,5-bis-trifluoromethyl-phenyl) -N-{6-[(2-hydroxy-ethyl) -methyl-amino]-4-o-tolyl-pyridin-3-yl}-N-methyl-isobutyramide,

(R) -2-(3,5-bis-trifluoromethyl-phenyl) -N-[6-(3-hydroxy-pyrrolidin-1-yl) -4-o-tolyl-pyridin-3-yl]-N-methyl-isobutyramide,

2-(3,5-bis-trifluoromethyl-phenyl)-N-methyl-N-(6-morpholin-4-yl-4-o-tolyl-pyridin-3-yl)-acetamide, and

[2-(3,5-bis-trifluoromethyl-phenyl)-2-methyl-propyl]- 8 4-(4-fluoro-2-methyl-phenyl)-6-(4-methyl-piperazin-1-yl) -pyridin-3-yl]-methylamine.

The methods for the preparation of the above-mentioned compounds is described in detail in EP-A-1,035,115. Also provided are values for the affinity of selected compounds to the NK-1 receptor, given as pKi, whereby the pKi value for preferred compounds is in the range of 8.00 to 9.80. EP-A-1,035,115 provides furthermore proposals for suitable formulations of NK-1 receptor antagonists, which are also suitable for the use as claimed in the present patent specification.

Most preferred compound for the use in accordance with the present invention are 2-(3,5-bis-trifluoromethyl-phenyl)-N-methyl-N-(6-morpholin-4-yl-4-o-tolyl-pyridin-3-yl)-isobutyramide and 2-(3,5-bis-trifluoromethyl-phenyl)-N-methyl-N-[6-(4-methyl-piperazin-1-yl)-4-o-tolyl-pyridin-3-yl]-isobutyramide disclosed in EP-A-1,035,115, as well as 2-(3,5-bis-trifluoromethyl-phenyl)-N-[6-( 1,1-dioxo-1λ⁶-thiomorpholin-4-yl)-4-o-tolyl-pyridin-3-yl]-N-methyl-isobutyramide and 2-(3,5-bis-trifluoromethyl-phenyl)-N-[6-(1,1-dioxo-1λ⁶-thiomorpholin-4-yl)-4-(4-fluoro-2-methyl-phenyl)-pyridin-3-yl]-N-methyl-isobutyramide described in European Patent Application 01118412.4 filed Jul. 31, 2001.

Methods for the preparation of additional compounds falling within the scope of general formula (I), which compounds are also suitable for the claimed uses are described in International Patent Application No. PCT/EP01/08432 filed Jul. 7, 2001 based on European Patent Application No. 00115846.8 filed Jul. 24, 2000. Examples of such compounds are:

A) compound of general formula (I), in which X is -C(O)N(R⁵)- and R⁵ is methyl, ethyl or cyclopropyl such as:

N-(3,5-bis-trifluoromethyl-benzyl)-N-methyl-4-o-tolyl-6- [1,2,4]triazol-1-yl-nicotinamide,

N-(3,5-bis-trifluoromethyl-benzyl)-6-(2-hydroxy-ethylamino)-N-methyl-4-o-tolyl-nicotinamide,

4-hydroxy-4′-o-tolyl-3,4,5,6-tetrahydro-2H- [1,2′]bipyridinyl-5′-carboxylic acid (3,5-bis-trifluoromethyl-benzyl) -methyl-amide,

4-(2-hydroxy-ethoxy)-4′-o-tolyl-3,4,5,6-tetrahydro-2H- [1,2′]bipyridinyl-5′-carboxylic acid (3,5-bis-trifluoromethyl-benzyl)-methyl-amide,

(R)-N-(3,5-bis-trifluoromethyl-benzyl)-6-(3-hydroxy-pyrrolidin-1-yl)-N-methyl-4-o-tolyl-nicotinamide,

4′-(2-chloro-phenyl)-4-hydroxy-3,4,5,6-tetrahydro-2H- [1,2′]bipyridinyl-5′-carboxylic acid (3,5-bis-trifluoromethyl-benzyl)-methyl-amide

B) compound of general formula (I), in which X is -N(R⁵)-C(O)- and R⁵ is hydrogen or methyl such as:

2-(3,5-bis-trifluoromethyl-phenyl)-N- [6-(2-hydroxy-ethylamino)-4-o-tolyl-pyridin-3-yl]-N-methyl-isobutyramide,

2-(3,5-bis-trifluoromethyl-phenyl)-N- [6-(2,3-dihydro- [1,4] oxazin-4-yl)-4-o-tolyl-pyridin-3-yl]-N-methyl-isobutyramide,

N-(6-acetylamino-4-o-tolyl-pyridin-3-yl)-2-(3,5-bis-trifluoromethyl-phenyl)-N-methyl-isobutyramide,

N-[6-(acetyl-methyl-amino)-4-o-tolyl-pyridin-3-yl]-2-(3,5-bis-trifluoromethyl-phenyl)-N-methyl-isobutyramide,

cyclopropanecarboxylic acid (5-{[2-(3,5-bis-trifluoromethyl-phenyl)-2-methyl-propionyl]-methyl-amino}-4-o-tolyl-pyridin-2-yl)-amide,

cyclopropanecarboxylic acid (5-{[2-(3,5-bis-trifluoromethyl-phenyl)-2-methyl-propionyl]-methyl-amino}-4-o-tolyl-pyridin-2-yl) -methyl-amide,

2-(3,5-bis-trifluoromethyl-phenyl)-N- (6-imidazol-1-yl-4-o-tolyl-pyridin-3-yl)-N-methyl-isobutyramide; or

2-(3,5-bis-trifluoromethyl-phenyl)-N- [4-(2-chloro-phenyl)-6-(2-hydroxy-ethylamino)-pyridin-3-yl]-N-methyl-isobutyramide.

Also preferred are compounds according to formula (I), wherein R⁴ is -(C═-C)ₙR⁷ or -(CR′═CR″)ₙR⁷. Typical compounds in this group can be characterized as follows:

Compounds of formula (I), in which X is -C(O)N(CH₃)- and -(R²)ₙ is 3,5-di-CF₃ represent a first group of compounds. Exemplary preferred compounds of this group are those, wherein R³/R³ are both hydrogen and R is methyl, for example the following compounds:

N-(3,5-bis-trifluoromethyl-benzyl)-6-(4-hydroxyacetyl-piperazin-1-yl)-N-methyl-4-o-tolyl-nicotinamide,

N-(3,5-bis-trifluoromethyl-benzyl) -6-chloro-N-methyl-4-o-tolyl-nicotinamide,

N-(3,5-bis-trifluoromethyl-benzyl)-6-cyanomethyl-N-methyl-4-o-tolyl-nicotinamide,

N-(3,5-bis-trifluoromethyl-benzyl)-6-iodo-N-methyl-4-o-tolyl-nicotinamide,

4-o-tolyl-[2,4′]bipyridinyl-5-carboxylic acid (3,5-bis-trifluoromethyl-benzyl)-methyl-amide,

5-[(3 ,5-bis-trifluoromethyl-benzyl) -methyl-carbamoyl]-4-o-tolyl-pyridine-2-carboxylic acid methyl ester,

N-(3,5-bis-trifluoromethyl-benzyl)-6-hydroxymethyl-N-methyl-4-o-tolyl-nicotinamide,

6- ( 5-acetyl-thiophen-2-yl) -N- (3,5-bis-trifluoromethyl-benzyl) -N-methyl-4-o-tolyl-nicotinamide,

4-o-tolyl-1′,2′,3′,6′-tetrahydro- [2,4′]bipyridinyl-5-carboxylic acid (3,5-bis-trifluoromethyl-benzyl) -methyl-amide,

N-(3,5-bis-trifluoromethyl-benzyl)-6-(4-hydroxymethyl-phenyl)-N-methyl-4-o-tolyl-nicotinamide,

2′-methyl-4-o-tolyl-[2,4′]bipyridinyl-5-carboxylic acid (3,5-bis-trifluoromethyl-benzyl) -methyl-amide,

N-(3,5-bis-trifluoromethyl-benzyl)-N-methyl-6-(3-methyl- [1,2,4] oxadiazol-5-yl)-4-o-tolyl-nicotinamide,

6-(3-amino-prop-1-ynyl)-N-(3,5-bis-trifluoromethyl-benzyl)-N-methyl-4-o-tolyl-nicotinamide,

(RS)-N-(3,5-bis-trifluoromethyl-benzyl)-6-(2-hydroxy-ethanesulfinylmethyl)-N-methyl-4-o-tolyl-nicotinamide,

N-(3,5-bis-trifluoromethyl-benzyl)-N-methyl-6-(1-methyl-1lH-imidazol-2-yl-sulfanylmethyl)-4-o-tolyl-nicotinamide,

(RS)-N-(3,5-bis-trifluoromethyl-benzyl)-N-methyl-6-(pyridine-2-sulfinyl)-4-o-tolyl-nicotinamide,

N-(3,5-bis-trifluoromethyl-benzyl)-N-methyl-6-(pyridine-2-sulfonyl)-4-o-tolyl-nicotinamide or

N-(3,5-bis-trifluoromethyl-benzyl)-6-(3-hydroxy-propoxy)-N-methyl-4-o-tolyl-nicotinamide.

Further preferred are compounds of formula (I) wherein R⁴ is -(C=-C)ₙR⁷ or -(CR′═CR″)ₙR⁷ and in which X is -N(CH₃)C(O)- and -(R²)n is 3,5-di-CF₃. Exemparly preferred compounds of this group are those, wherein R³/R³ are both methyl and R is methyl, for example the following compounds:

2- (3,5-bis-trifluoromethyl-phenyl)-N-{6- [hydroxy-(2-hydroxy-ethyl) -amino]-4-o-tolyl-pyridin-3-yl}-N-methyl-isobutyramide,

2-(3,5-bis-trifluoromethyl-phenyl)-N-methyl-N-[6-(3-oxo-morpholin-4-yl)-4-o-tolyl-pyridin-3-yl]-isobutyramide,

acetic acid (5-{[2-(3,5-bis-trifluoromethyl-phenyl)-2-methyl-propionyl]-methyl-amino}-4-o-tolyl-pyridin-2-ylcarbamoyl)-methyl ester,

2-(3,5-bis-trifluoromethyl-phenyl)-N-[6-(2-hydroxy-acetylamino)-4-o-tolyl-pyridin-3-yl]-N-methyl-isobutyramide,

2-(3,5-bis-trifluoromethyl-phenyl)-N-[6-(hydroxyacetyl-methyl-amino)-4-o-tolyl-pyridin-3-yl]-N-methyl-isobutyramide,

2-(3,5-bis-trifluoromethyl-phenyl)-N- [6-(2,5-dioxo-pyrrolidin-1-yl)-4-o-tolyl-pyridin-3-yl]-N-methyl-isobutyramide,

cyclopropanecarboxylic acid (5-{[2-(3,5-bis-trifluoromethyl-phenyl)-2-methyl-propionyl]-methyl-amino}-4-o-tolyl-pyridin-2-yl)-cyclopropanecarbonyl-amide,

2-(3,5-bis-trifluoromethyl-phenyl)-N-(6-chloro-4-o-tolyl-pyridin-3-yl)-N-methyl-isobutyramide,

2- (3,5-bis-trifluoromethyl-phenyl) -N- [4- (2-chloro-phenyl) -2′-methyl-[2,4′]bipyridinyl-5-yl]-N-methyl-isobutyramide,

2-(3,5-bis-trifluoromethyl-phenyl)-N-(6-ethynyl-4-o-tolyl-pyridin-3-yl)-N-methyl-isobutyramide,

2-(3,5-bis-trifluoromethyl-phenyl)-N-[6-(3-hydroxymethyl-isoxazol-5-yl)-4-o-tolyl-pyridin-3-yl]-N-methyl-isobutyramide,

2-(3,5-bis-trifluoromethyl-phenyl) -N- [6- (3-hydroxy-prop-1-ynyl) -4-o-tolyl-pyridin-3-yl]-N-methyl-isobutyramide or

(RS)-2-(3,5-bis-trifluoromethyl-phenyl)-N- [6-(3-methoxy-benzenesulfinyl)-4-o-tolyl-pyridin-3-yl]-N-methyl-isobutyramide.

Further preferred compounds of formula (I) wherein R⁴ is -(C═C)ₙR⁷ or (CR′═CR″)ₙR⁷ are those, wherein R³/R³ are both methyl and R is chloro, for example the following compounds:

2-(3,5-bis-trifluoromethyl-phenyl)-N-{4-(2-chloro-phenyl)-6- [hydroxy-(2-hydroxy-ethyl)-amino]-pyridin-3-yl}-N-methyl-isobutyramide, or

2- (3,5-bis-trifluoromethyl-phenyl) -N- [4- (2-chloro-phenyl) -6- (3-oxo-morpholin-4-yl)-pyridin-3-yl]-N-methyl-isobutyramide.

The methods for the preparation of the compounds of formula (I) wherein R⁴ is -(C═-C)ₙR⁷ or -(CR′═CR″)nR⁷ are described in detail in International Patent Application No. PCT/EPO1/08686 filed Jul. 27, 2001 based on European Patent Application No. 00117003.4 filed Aug. 8, 2000.

As indicated above the NK-1 receptor antagonist in accordance with the use of the present invention may be present in the form of a prodrug.

Preferred prodrugs of the compounds of general formula (I) are N-oxides such as the following exemplary compounds:

4-{5-[(3,5-bis-trifluoromethyl-benzyl) -methyl-carbamoyl]-4-o-tolyl-pyridin-2-yl}-4-oxy-piperazine-1-carboxylic acid tert-butyl ester,

5′-[(3,5-bis-trifluoromethyl-benzyl) -methyl-carbamoyl]-4′-o-tolyl-1-oxy-3,4,5,6-tetrahydro-2H- [1,2′]bipyridinyl-4-carboxylic acid ethyl ester,

(RS)-6- 8 3-(acetyl-methyl-amino)-1-oxo-pyrrolidin-1-yl]-N-(3,5-bis-trifluoromethyl-benzyl) -N-methyl-4-o-tolyl-nicotinamide,

N-(3,5-bis-trifluoromethyl-benzyl)-N-methyl-6-(4-oxy-morpholin-4-yl)-4-o-tolyl-nicotinamide,

N-(3,5-bis-trifluoromethyl-benzyl)-6-(1,1-dioxo-1λ⁶-4-oxy-thiomorpholin-4-yl)-N-methyl-4-o-tolyl-nicotinamide,

N-(3,5-bis-trifluoromethyl-benzyl)-6-(4-formyl-1-oxy-piperazin-1-yl) -N-methyl-4-o-tolyl-nicotinamide,

N-methyl-N-(2-methyl-naphthalen-1-yl-methyl)-6-(4-oxy-morpholin-4-yl)-4-o-tolyl-nicotinamide,

N-methyl-6-(4-oxy-morpholin-4-yl)-N-naphthalen-1-yl-methyl-4-o-tolyl-nicotinamide,

N-(2-methoxy-naphthalen-1-yl-methyl)-N-methyl-6-(4-oxy-morpholin-4-yl)-4-o-tolyl-nicotinamide,

N-(2-methoxy-benzyl)-N-methyl-6-(4-oxy-morpholin-4-yl)-4-o-tolyl-nicotinamide,

N-(5-chloro-2-methoxy-benzyl)-N-methyl-6-(4-oxy-morpholin-4-yl)-4-o-tolyl-nicotinamide,

N-(2-chloro-5-methoxy-benzyl)-N-methyl-6-morpholin-4-yl-4-o-tolyl-nicotinamide,

N-methyl-6-(4-oxy-morpholin-4-yl)-N-pentafluorophenylmethyl-4-o-tolyl-nicotinamide,

N-methyl-6-(4-oxy-morpholin-4-yl)-N-naphthalen-2-yl-methyl-4-o-tolyl-nicotinamide,

N-[2-methoxy-5-(5-trifluoromethyl-tetrazol-1-yl)-benzyl]-N-methyl-6-(4-oxy-morpholin-4-yl)-4-o-tolyl-nicotinamide,

N-(1,4-dimethoxy-naphthalen-2-yl-methyl)-N-methyl-6-(4-oxy-morpholin-4-yl)-4-o-tolyl-nicotinamide,

5′-[(3,5-bis-trifluoromethyl-benzyl) -methyl-carbamoyl]-4′-o-tolyl-1-oxy-3,4,5,6-tetrahydro-2H-[1,2′]bipyridinyl-4-carboxylic acid,

2-(3,5-bis-trifluoromethyl-phenyl)-N-methyl-N-[6-(4-oxy-morpholin-4-yl)-4-o-tolyl-pyridin-3-yl]-isobutyramide,

2-(3,5-bis-trifluoromethyl-phenyl)-N-[4-(2-chloro-phenyl)-6-(4-oxy-morpholin-4-yl)-pyridin-3-yl]-N-methyl-isobutyramide,

2-(3,5-bis-trifluoromethyl-phenyl)-N-[6-(4-oxy-morpholin-4-yl) -4-o-tolyl-pyridin-3-yl ]-isobutyramide,

2-(3,5-bis-trifluoromethyl-phenyl)-N-[4′-(2-chloro-phenyl)-1-oxy-3,4,5,6-tetrahydro-2H- [1,2′]bipyridinyl-5′-yl]-N-methyl-isobutyramide,

2-(3,5-bis-trifluoromethyl-phenyl)-N-(6-oxy-dimethylamino-4-o-tolyl-pyridin-3-yl)-N-methyl-isobutyramide,

2-(3,5-bis-trifluoromethyl-phenyl)-N-[4-(2-chloro-phenyl)-6-oxy-dimethylamino-pyridin-3-yl]-isobutyramide,

2-(3,5-bis-trifluoromethyl-phenyl)-N-1-(4-hydroxy-1-oxy-4′-o-tolyl-3,4,5,6-tetrahydro-2H- [1,2′]bipyridinyl-5′-yl)-N-methyl-isobutyramide,

2-(3,5-bis-trifluoromethyl-phenyl)-N-{6- [(2-hydroxy-ethyl) -1-oxy-methyl-amino]-4-o-tolyl-pyridin-3-yl}-N-methyl-isobutyramide,

(R)-2-(3,5-bis-trifluoromethyl-phenyl)-N-[6-(3-hydroxy-1-oxy-pyrrolidin-1-yl)-4-o-tolyl-pyridin-3-yl]-N-methyl-isobutyramide,

2-(3,5-bis-trifluoromethyl-phenyl)-N-methyl-N-[6-(4-oxy-morpholin-4-yl) -4-o-tolyl-pyridin-3-yl]-acetamide,

2-(3,5-dimethoxy-phenyl)-N-methyl-N-[6-(4-oxy-morpholin-4-yl)-4-o-tolyl-pyridin-3-yl]-acetamide; or

2-(3-fluoro-5-trifluoromethyl-phenyl)-N-methyl-N-[6-(4-oxy-morpholin-4-yl)-4-o-tolyl-pyridin-3-yl]-acetamide.

Methods for the preparation of the above-mentioned N-oxide prodrugs are described in International Patent Application No. PCT/EPO1/07850 filed Jul. 9, 2001 based on European Patent Application No. 00115287.5 filed Jul. 14, 2000. The most preferred N-oxide prodrug of general formula (I) for the claimed use is 2-(3,5-bis-trifluoromethyl-phenyl)-N-methyl-N-[6-(4-oxy-morpholin-4-yl)-4-o-tolyl-pyridin-3-yl]-isobutyramide.

Other suitable NK-1 receptor antagonists are described in the following patent publications:

International Patent Application, WO 00/50398 of Boes M., Galley G., Godel T., Hoffmann T., Hunkeler W., Schnider P., Stadler H., entitled “Preparation of phenyl and pyridinyl derivatives as NK-1 receptor antagonists.”

International Patent Publication No. WO 00/50401 of Boes M., Galley G., Godel T., Hoffmann T., Hunkeler W., Schnider P., Stadler H., entitled “Preparation of 3-phenylpyridines as NK-1 receptor antagonists.”

International Patent Publication No. WO 00/53572 of Boes M., Galley G., Godel T., Hoffmann T., Hunkeler W., Schnider P., Stadler H., entitled “Preparation of biphenyl derivatives as antagonists of the neurokinin-1 receptor.”

International Patent Publication No. WO 00/73278 of Boes M., Galley G., Godel T., Hoffmann T., Hunkeler W., Schnider P., Stadler H., entitled “Novel 5-phenyl-pyrimidine derivatives as NK-1 receptor antagonists.”

International Patent Publication No. WO 00/73279 of Boes M., Galley G., Godel T., Hoffmann T., Hunkeler W., Schnider P., Stadler H., entitled “Novel 4-phenyl-pyrimidine derivatives as NK-1 receptor antagonists.”

International Patent Application No. PCT/EP01/05723 filed May 18, 2001.

International Patent Application No. PCT/EP01/06305 filed Jun. 1, 2001.

International Patent Application No. PCT/EP01/13084 filed Nov. 13, 2001.

European Patent Application No. 01102557.4 filed Feb. 6, 2001.

Further preferred NK-1 receptor antagonists useful in connection with the present invention are the following NK-1 receptor antagonists some of which are currently under drug development:

GR205171: 3-Piperidinamine, N-[[2-methoxy-5- [5-(trifluoromethyl)- lH-tetrazol-1-yl]phenyl]methyl]-2-phenyl-, (2S-cis)- (Gardner et al. Regul. Pep. 65:45, 1996)

HSP-117: 3-Piperidinamine, N-[[2,3-dihydro-5-(1-methylethyl)-7-benzofuranyl]methyl]-2-phenyl-, dihydrochloride, (2S-cis)-

L 703,606: 1-Azabicyclo[2.2.2] octan-3-amine, 2-(diphenylmethyl)-N-[(2-iodophenyl)methyl]-, (2S-cis)-, oxalate (Cascieri et al., Mol. Pharmacol. 42, 458, 1992)

L 668,169: L-Phenylalanine, N-[2-[3-[[N-[2-(3-amino-2-oxo-1-pyrrolidinyl)-4-methyl-1- oxopentyl]-L-methionyl-L-glutaminyl-D-tryptophyl-N-methyl-L-phenylalanyl]amino]-2-oxo-1-pyrrolidinyl]-4-methyl-i-oxopentyl]-L-methionyl-L-glutaminyl-D-tryptophyl-N-methyl-,cyclic (8->1)-peptide, [3R-[1[S*[R*(S*)]],3R*]]-

LY 303241: 1-Piperazineacetamide, N-[2-[acetyl[(2-methoxyphenyl)methyl]amino]-1-(1H- indol-3-yl-methyl)ethyl]-4-phenyl-, (R)-

LY 306740: 1-Piperazineacetamide, N- [2- [acetyl[(2-methoxyphenyl)methyl]amino]-1-(1H- indol-3-yl-methyl)ethyl]-4-cyclohexyl-, (R)-

MK-869: 3H-1,2,4-Triazol-3-one, 5- [[2-[1-[3,5-bis(trifluoromethyl)phenyl]ethoxy]-3-(4-fluorophenyl) -4-morpholinyl]methyl]-1,2-dihydro-, [2R- [2α(R*) ,3α]]-

R-544: Ac-Thr-D-Trp(FOR)-Phe-N-MeBzl

Spantide III: L-Norleucinamide, N6-(3-pyridinylcarbonyl)-D-lysyl-L-prolyl-3-(3-pyridinyl)-L-alanyl-L-prolyl-3,4-dichloro-D-phenylalanyl-L-asparaginyl-D-tryptophyl-L-phenylalanyl-3-(3-pyridinyl)-D-alanyl-L-leucyl-

WIN-62,577: 1H-Benzimidazo[2,1-b] cyclopenta[5,6] naphtho [1,2-g] quinazolin-1-ol, 1-ethynyl-2,3,3a,3b,4,5,15,15a,15b,16,17,17a-dodecahydro-15a,17a-dimethyl-,(1R,3aS,3bR,15aR,15bS,17aS)-

GR 103,537

L 758,298: Phosphonic acid, [3-[[2-[1-[3,5-bis(trifluoromethyl)phenyl]ethoxy]-3-(4-fluorophenyl)-4-morpholinyl]methyl]-2,5-dihydro-5-oxo-1H-1,2,4-triazol-1-yl]-, [2R- [2α(R*),3α]]-

NKP608: (2R,4S)-N- [1- {3,5-bis(trifluormethyl)-benzoyl}-2- (4-chloro-benzyl) -4-piperidinyl]-quinoline-4-carboxamide

CGP49823: (2R, 4S) -2-benzyl-1-(3, 5-dimethylbenzoyl) -N- [(4-quinolinyl)methyl]-4-piperineamine) dihydrochloride

CP-96,345: (2S, 3S)-cis-(2(diphenylmethyl)-N- [(2-methoxyphenyl)methyl]-1-azabicyclo[2.2.2]octan-3-amine (Srider et al., Science 251:435, 1991)

CP-99,994: ((2S, 3S)-cis-3-(2-methoxybenzylamino)-2-phenyl-piperidine)dihydrochloride (Desai et al., J. Med. Chem. 35:4911, 1992)

CP-122,721: (+)-(2S, 3S)-3-(2-methoxy-5-trifluoromethoxybenzyl)amino-2-phenylpiperidine

FK 888: (N2-[(4R)-4-hydroxy-1(1-methyl-1H-indol-3-yl)cabonyl-L-propyl-N-methyl-N-phenylmethyl-L-3-(2-naphthyl)-alaninamide (Fujii et al., Br. J. Pharm. 107:785, 1992)

GR203040: (2S, 3S and 2R, 3R)-2methoxy-5-tetrazol-1-yl-benzyl-(2-phenyl-piperidin-3-yl)-amine

GR 82334: [D-Pro9,][spiro-gamma-lactam][Leu10,Trp11]physalaemin-(1-11)

GR 94800: PhCO-Ala-Ala-DTrp-Phe-DPe-DPro-Pro-NIe-NH2

L 732,138: N-acetyl-L-tryptophan

L 733,060: (2S,S)-3-((3,5-bis(trifluoromethyl)phenyl)methyloxy)-2-phenyl piperidine

L 742,694: (2-(S)-(3,5-bis(trifluromethyl)benzyloxy)-3-(S)-phenyl-4-(3-oxo-1, 2, 4-triazolo)methylmorpholine

L 754,030: 2-(R)-(1-(R)-3,5-bis(trifluoromethyl)phenylethoxy)-3-(S)- (4-fluoro)phenyl-4-(3-oxo-1,2,4-triazol-5-yl)methylmorpholine

LY 303870: (R)-1[N-(2-methoxybenzyl)acetylamino]-3-(1H-indol-3-yl)-2-[N-(2-(4-(pi-peridinyl)piperidin-1-yl)acetyl)amino]propane

MEN 11149: 2-(2-naphthyl)-1-N-[(lR, 2S)-2-N-[2(H)indol-3-ylcarbonyl]aminocyclohexanecarbonyl]-1-[N′-ethyl-N′-(4methylphenylacetyl)]diaminoethane (Cirillo et al., Eur. J. Pharm. 341:201, 1998)

PD 154075: (2-benzofuran)-CH20CO]-(R)-alpha-MeTrp-(S)-NHCH(CH3) Ph

RP-67580: (3aR, 7aR) -7, 7-diphenyl-2 [1-imino-2 (2-methoxyphenyl)-ethyl]perhydroisoidol-4-one hydrochloride (Garret et al., PNAS 88:10208, 1991)

RPR 100893: (3aS, 4S, 7aS)-7, 7-diphenyl-4-(2-methoxyphenyl)-2- [(S)-2-(2-methoxyphenyl)proprionyl]perhydroisoindol-4-ol

Spendide: Tyr-D-Phe-Phe-D-His-Leu-Met-NH2

Spantide II: D-NicLysl, 3-PaI3, D-CI2Phe5, Asn6, D-Trp7.0, NIel 1-substance P

SR140333: (S)-1- [2- [3-(3, 4-dichlorphenyl)-1 (3-isopropoxyphenylacetyl) piperidin-3-yl]ethyl]-4-phenyl-1 azaniabicyclo [2.2.2]octane (Edmonts et al., Eur. J. Pharm. 250:403, 1993)

WIN-41,708: 17beta-hadroxy-17alpha-ethynyl-5alpha-androstano[3.2-b]pyrimido [1,2-a]benzimidazole

WIN-62,577: 1H-Benzimidazo [2,1-b]cyclopenta [5,6]naphtho [1,2-g]quinazolin-1-ol, 1-ethynyl-2,3,3a,3b,4,5,15,15a,15b,16,17,17a-dodeachydro-15a,17a-dimethyl-,( IR, 3aS, 3bR,15aR, 15bS, 17aS)-

SR-48,968: (S)-N-methyl-N[4-(4-acetylamino-4-phenylpiperidino)-2-(3,4-dichlorophenyl)-butyl]benzamide

L-659,877: cyclo[Gln, Trp, Phe, Gly, Leu, Met]

MEN 10627: cyclo(Met-Asp-Trp-Phe-Dap-Leu)cyclo(2beta-5beta)

SR 144190: (R)-3(1- [2-(4-benzoyl-2-(3,4-difluorophenyl)-morpholin-2-yl)ethyl]-4-phenylpiperidin-4-yl) -1-dimethylurea

GR 94800: PhCO-Ala-Ala-D-Trp-Phe-D-Pro-Pro-NIe-NH2

SR-142,801: (S)-(N)-(1 -(3-(1-benzoyl-3-(3,4-dichlorophenyl)piperidin-3-yl)propyl)-4-phenylpiperidin-4-yl)-N-methyl acetamide

R820: 3-indolylcarbonyl-Hyp-Phg-N(Me)-Bzl

R486: H-Asp-Ser-Phe-Trp-beta-Ala-Leu-Met-NH2

SB 222200: (S) - ( -) -N- (a-ethylbenzyl) -3-methyl-2-phenylquinoline-4-carboximide

L 758,298: Phosphonic acid, [3-[2-[1-[3, 5-bis(trifluoromethyl)phenyl]ethoxy]-3-(4-fluorophenyl) -4-morpholinyl]-2, 5-dihydro-5oxo-1H-1,2,4-triazol-1-yl]-, [2R- [2a(R*), 3a]]-

NK-608: (2R,4S)-N- [1-{3,5-bis(trifluormethyl)-benzoyl}-2-(4-chloro-benzyl)-4-piperidinyl]- quinoline-4-carboxamide

CGP 47899: Shilling et al., Pers. Med. Chem. 207, 1993

MEN 11467: Evangelista et al., XXIX Nat. Congr. of the Ital. Pharmacological Soc., Florence 20-23.06.1999.

Any reference herein to the compound specifically named above includes also the pharmaceutically acceptable acid addition salts thereof.

Further information on these NK-1 receptor antagonists under drug development can be found in the published literature.

Additional suitable NK-1 receptor antagonists are described in the following published patents and patent applications.

U.S. Pat. No. 5,990,125 in particular the compounds Ia to Ie, X and XVI to XXI, as well as other antagonists comprising quinuclidine, piperidine ethylene diamine, pyrrolidine and azabornane derivatives and related compounds that exhibit activity as substance P receptor antagonists as described in column 33 of U.S. Pat. No. 5,990,125. These antagonists are preferably used in dosages as specified in column 34 of U.S. Pat. No. 5,990,125.

Further suitable NK-1 receptor antagonists are described in the following publications:

U.S. Pat. Nos. (USP)

| | | | |
|---|---|---|---|
| 5,977,104 | 5,162,339 | 4,481,139 | 5,232,929 |
| 5,998,444 | 5,242,930 | 5,373,003 | 5,981,744 |
| 5,387,595 | 5,459,270 | 5,494,926 | 5,496,833 |
| 5,637,699 | | | |

Europ. Patent Application, Publ. Nos. (EP-A-)

| | | | |
|---|---|---|---|
| 0 360 390 | 0 394 989 | 0 428 434 | 0 429 366 |
| 0 430 771 | 0 436 334 | 0 433 132 | 0 482 539 |
| 0 498 069 | 0 499 313 | 0 512 901 | 0 512 902 |
| 0 514 273 | 0 514 274 | 0 514 275 | 0 514 276 |
| 0 515 681 | 0 517 589 | 0 520 555 | 0 522 808 |
| 0 528 495 | 0 532 456 | 0 533 280 | 0 536 817 |
| 0 545 478 | 0 558 156 | 0 577 394 | 0 585 913 |
| 0 590 152 | 0 599 538 | 0 610 793 | 0 634 402 |
| 0 686 629 | 0 639 489 | 0 694 535 | 0 699 655 |
| 0 699 674 | 0 707 006 | 0 708 101 | 0 709 375 |
| 0 709 376 | 0 714 891 | 0 723 959 | 0 733 632 |
| 0 776 893 | | | |

PCT Int. Patent Publ. Nos. (WO)

| | | | |
|---|---|---|---|
| 90/05525 | 90/05729 | 91/09844 | 91/18899 |
| 92/01688 | 92/06079 | 92/12151 | 92/15585 |
| 92/17449 | 92/20661 | 92/20676 | 92/21677 |
| 92/22569 | 93/00330 | 93/00331 | 93/01159 |
| 93/01165 | 93/01169 | 93/01170 | 93/06099 |
| 93/09116 | 93/10073 | 93/14084 | 93/14113 |
| 93/18023 | 93/19064 | 93/21155 | 93/21181 |
| 93/23380 | 93/24465 | 94/00440 | 94/01402 |
| 94/02461 | 94/02595 | 94/03429 | 94/03445 |
| 94/04494 | 94/04496 | 94/05625 | 94/07843 |
| 94/08997 | 94/10165 | 94/10167 | 94/10168 |
| 94/10170 | 94/11368 | 94/13639 | 94/13663 |
| 94/14767 | 94/15903 | 94/19320 | 94/19323 |
| 94/20500 | 94/26735 | 94/26740 | 94/29309 |
| 95/02595 | 95/04040 | 95/04042 | 95/06645 |
| 95/07886 | 95/08908 | 95/08549 | 95/11880 |
| 95/14017 | 95/15311 | 95/16679 | 95/17382 |
| 95/18124 | 95/18129 | 95/19344 | 95/20575 |
| 95/21819 | 95/22525 | 95/23798 | 95/26338 |
| 95/28418 | 95/30674 | 95/30687 | 95/33744 |
| 96/05181 | 96/05193 | 96/05203 | 96/06094 |
| 96/07649 | 96/10562 | 96/16939 | 96/18643 |
| 96/20197 | 96/21661 | 69/29304 | 96/29317 |
| 96/29326 | 96/29328 | 96/31214 | 96/32385 |
| 96/37489 | 97/01553 | 97/01554 | 97/03066 |
| 97/08144 | 97/14671 | 97/17362 | 97/18206 |
| 97/19084 | 97/19942 | 97/21702 | 97/49710 |

British Patent Publ. Nos. (GB)

| | | | |
|---|---|---|---|
| 2 266 529 | 2 268 931 | 2 269 170 | 2 269 590 |
| 2 271 774 | 2 292 144 | 2 293 168 | 2 293 169 |
| 2 302 689 | | | |

As mentioned above benign prostatic hyperplasia (BPH) or prostate hypertrophy is a disease of males, the incidence of which increases considerably after the fifth decade in the life of human beings. It is still not clear what causes BPH, but it appears that BPH is related to the hormone testosterone and its relationship to other hormones that change during the aging process. The fact that the prostate begins to grow larger is not necessarily a problem. In fact, some men have extremely enlarged prostates but suffer no ill effects. On the other hand, some men have prostates that are only slightly enlarged and they suffer from bothersome urinary symptoms.

These symptoms include difficulties in urinating, the need to urinate quite frequently, or awaking during the night to urinate.

In serious cases BPH will either be treated through medical therapy using prescription medications or by surgical treatment to remove tissue that is obstructing the flow of urine. Therapy by prescription medication is preferred because it is non-invasive. A number of prescription medications for the treatment of BPH are known, such as e.g. the gonadotrophin agonist leuprorelin sold inter alia under the tradenames Lupron™ and Lupron Depot™ and the 5-alpha reductase inhibitor finasteride sold under the trademark of Proscar™. The present invention provides a novel class of prescription medication for the treatment of BPH, viz. NK-1 receptor antagonists.

NK-1 receptor antagonist for use in connection with the claimed invention may be administered either alone or in combination with other therapeutic agents and are preferably formulated to a pharmaceutical composition comprising pharmaceutically acceptable carriers or diluents. The pharmaceutical preparations to be used in accordance with this invention can in addition also contain preservatives, solubilizers, stabilizers, wetting agents, emulsifiers, sweeteners, colorants, flavorants, salts for varying the osmotic pressure, buffers, masking agents or antioxidants.

NK-1 receptor antagonists can be formulated in the form of a Self-Emulsifying Drug Delivery Systems (SEDDS), which consist of mixtures of oils and surfactants, ideally isotropic, which sometimes include co-solvents. Such mixtures emulsify under conditions of gentle agitation, similar to those which would be encountered in the gastro intestinal tract. When such a formulation is released into the lumen of the gut, it disperses to form a fine emulsion, so that the drug contained in the emulsion remains in solution in the gut, avoiding the dissolution step which frequently limits the rate of absorption of hydrophobic drugs from the crystalline state. SEDDS lead to improved bioavailability and/or a more consistent temporal profile of absorption from the gut. SEDDS have been described by Pouton C. W., in Advanced Drug Delivery Reviews, 25, (1997), 47-58.

The NK-1 receptor antagonist or the pharmaceutical composition comprising it is preferably administered orally, e.g. in the form of tablets, coated tablets, dragees, hard and soft gelatine capsules, solutions, emulsions or suspensions. The administration can, however, also be effected rectally, e.g. in the form of suppositories, or parenterally, e.g. in the form of injection solutions. The NK-1 receptor antagonist or the pharmaceutically composition comprising it can also be administered via any other suitable way known to the person skilled in the art.

The dosage can vary within wide limits and can, of course, be fitted to the individual requirements in each particular case. The dosage range for a beneficial effect in mammals depends of course on the activity of the NK-1 receptor antagonist that is used, but is usually in the range of 5 to 1000 mg/kg/d and is preferably between 25 and 100 mg/kg/d.

An injection solution may have the following composition:

| | |
|---|---|
| Compound of formula (I) | 1 mg |
| 1 n HCl | 20 μl |
| acetic acid | 0.5 mg |
| NaCl | 8 mg |
| phenol | 10 mg |
| 1 n NaOH | q.s. ad pH 5 |
| H₂O | q.s. ad 1 ml |

The pharmaceutical preparations in accordance with this invention can in addition also contain pharmaceutically inert, inorganic or organic excipients suitable for the production of tablets, coated tablets, dragees and hard gelatine capsules. Lactose, corn starch or derivatives thereof, talc, stearic acid or its salts etc. can be used as such excipients e.g. for tablets, dragees and hard gelatine capsules.

Suitable excipients for soft gelatine capsules are e.g. vegetable oils, waxes, fats, semi-solid and liquid polyols etc.

Suitable excipients for the manufacture of solutions and syrups are e.g. water, polyols, saccharose, invert sugar, glucose etc.

Suitable excipients for injection solutions are e.g. water, alcohols, polyols, glycerol, vegetable oils etc.

Suitable excipients for suppositories are e.g. natural or hardened oils, waxes, fats, semi- liquid or liquid polyols etc.

Moreover, the pharmaceutical preparations can contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

As indicated in the following example below the inventors have shown that NK-1 receptor antagonists, in particular 2-(3,5-bis-trifluoromethyl-phenyl)-N-methyl-N-(6-morpholin-4-yl-4-o-tolyl-pyridin-3-yl)-isobutyramide, have the potential to reduce the size of prostate and can therefore be used in the treatment and/or prevention of benign prostatic hyperplasia. While the following example illustrates the invention it is not meant to limit the scope of the claimed invention in any respect.

### EXAMPLES

### Determination of NK-1 Receptor Antagonist Binding Activity

The affinity of the compound of formula I for the NK₁ receptor was evaluated at human NK₁ receptors in CHO cells infected with the human NK₁ receptor (using the Semliki virus expression system) and radiolabelled with [³H] substance P (final concentration 0.6 nM). Binding assays were performed in HEPES buffer (50 mM, pH 7.4) containing BSA (0.04 %), leupeptin (8 μg /ml), MnCl₂ (3mM) and phosphoramidon (2 μM). Binding assays consisted of 250 μl of membrane suspension (1.25×10⁵ cells / assay tube), 0.125 μgl of buffer of displacing agent and 125 μl of [³H] substance P. Displacement curves were determined with at least seven concentrations of the compound. The assay tubes were incubated for 60 min at room temperature after which time the tube contents were rapidly filtered under vacuum through GF/C filters presoaked for 60 min with PEI (0.3%) with 2×2 ml washes of HEPES buffer (50 mM, pH 7.4). The radioactivity retained on the filters was measured by scintillation counting. All assays were performed in triplicate in at least 2 separate experiments.

### Summary on a 39-week Toxicity Study in the Dog

In a nine-month study four groups of Beagle dogs (4 animals/gender/group; 5-6 months of age at study start) received oral doses (gavage) of 0 (placebo), 6, 20 and 60 mg/kg/d of 2-(3,5-bis-trifluoromethyl-phenyl)-N-methyl-N-(6-morpholin-4-yl-4-o-tolyl-pyridin-3-yl)-isobutyramide as a SEDDS formulation for 39 weeks. The following variables were investigated: clinical signs, body weights, food consumption, ophthalmoscopy before and at the end of the study, electrocardiography, heart rate, toxicokinetics at different time-points, clinical pathology (hematology, biochemistry, urinalysis) in 3-months intervals, necropsy and tissue preservation, organ weights and histopathology.

Dose-related reduced weights of the prostate gland were measured in males at 60 and 20 mg/kg/d (by 58% and 37% when compared to the control) with an insignificant trend also at 6 mg/kg/d. Microscopic changes were limited to all males at 60 and most at 20 mg/kg/d.

The finding was characterized by a smaller overall cross-sectional area, smaller acinar lumina and flatter epithelium with less eosinophilic cytoplasm, particularly in the center of the prostate. However, mitoses were evident in the peripheral acini. The prostate of low dose males was similar to controls.

It is known that the canine prostate exhibits regional differences in the response of the prostatic epithelium to hormonal influences, the peri-urethral (central) glands being more sensitive to androgen withdrawal than sub-capsular (peripheral) zones, as occurred in this study. Thus the prostatic changes seen are considered to reflect a pharmacological effect of the compound used rather than evidence of toxicity.

Mean absolute organ weights were adjusted to 100 g of the mean terminal body weights=>mean relative organ weights.

Mean relative organ weights were compared to the corresponding control=>% deviation.

TABLE I

| | | | | PROSTATE | | |
|---|---|---|---|---|---|---|
| | TESTES | | | | per | |
| | | per 100 g | | | 100 g | |
| Dose | | BW | diff. % | | BW | diff. % |
| mg/kg/d | absolute | relative | vs. control | absolute | relative | vs. control |
| 0/veh | 24.006 | 0.1698 | (=100%) | 11.006 | 0.078 | (=100%) |
| 6 | 24.577 | 0.1803 | +1% | 8.066 | 0.059 | −24% |
| 20 | 23.432 | 0.1804 | +6% | 6.413 | 0.049 | −37% |
| 60 | 24.753 | 0.1853 | +9% | 4.429 | 0.033 | −58% |

Apart from the prostate changes no overt abnormalities in any other organ system was observed. Mild changes in the liver (hepatocyte hypertrophy with slightly increased organ weights) were considered to remain within the normal physiological adaptive range of dogs of this strain, with no signs of an overt systemic effect.

Preparation of 2-(3,5-Bis-trifluoromethyl-phenyl)-N-[6-(1,1-dioxo-1λ⁶-thiomorpholin-4-yl)-4-o-tolyl-pyridin-3-yl]-N-methyl-isobutyramide

a) 4-(5-Nitro-pyridin-2-yl)-thiomorpholine

To a solution of 20 g (126 mmol) of 2-chloro-5-nitropyridine in 200 ml tetrahydrofuran were added dropwise 32.5 ml (315 mmol) thiomorpholine within 10 min. The reaction mixture was refluxed for additional 2 h. After cooling to room temperature, the solvent was removed in vacuo and the residue was re-dissolved in 200 ml ethyl acetate. The organic phase was washed with 200 ml 1 N sodium bicarbonate solution, dried (magnesium sulfate) and evaporated to give 29.3 g (quantitative) of the title compound as a yellow solid.

MS m/e (%): 225 (M^{30,} 78), 152 (100), 124 (62).

b) 2,2-Dimethyl-N-(6-thiomorpholin-4-yl-pyridin-3-yl)-propionamide

To a suspension of 1.0 g (4.4 mmol) of 4-(5-nitro-2-pyridyl)-thiomorpholine in 8 ml ethanol and 2 ml water were added 1.5 g (27 mmol) of iron powder. A few drops of 3 N hydrochloric acid solution in diethyl ether were added and the reaction mixture was heated at 85° C. for 18 h. The suspension was filtered and the residue was washed 5 times with 10-ml portions of ethanol. The filtrate was evaporated in vacuo to give 870 mg of a purple oil.

This crude product was dissolved in 10 ml dichloromethane. Under stirring, 700 mg (6 mmol) of pivaloyl chloride and 860 mg (7 mmol) of N-ethyldiisopropylamine were added and the reaction mixture was stirred at room temperature overnight. Then, 30 ml water and 3 ml of 1 N hydrochloric acid solution were added to reach pH 1. The organic layer was separated and the aqueous layer was washed with 1 N hydrochloric acid solution, adjusted to pH 10 with sodium carbonate and extracted with dichloromethane. The organic layer was dried (sodium sulfate) and evaporated to give 630 mg (51 %) of the title compound as purple crystals.

MS m/e (%): 280 (M+H^{+,} 100).

c) N-(4-Iodo-6-thiomorpholin-4-yl-pyridin-3-yl)-2,2-dimethyl-propionamide Under argon, a solution of 75 g (268 mmol) 2,2-dimethyl-N-(6-thiomorpholin-4-yl-pyridin-3-yl)-propionamide, 187 g (1.61 mol) N,N,N′,N′-tetramethylethylenediamine and 85 g (604 mmol) 2,2,6,6,-tetramethylpiperidine in 750 ml tetrahydrofuran was cooled to −65° C. in a dry ice bath. Within 30 min, 805 ml (1.29 mol) of a 1.6 N n-butyllithium solution in hexane were added dropwise. The reaction mixture was allowed to warm up to −15° C. and was stirred for 3 h at this temperature. After cooling again to −70° C., 354 g (1.40 mol) iodine (dissolved in 1000 ml tetrahydrofuran) were added dropwise during 2 h and stirring was continued for 1 h. The suspension was warmed to −60° C. and was poured into 1000 ml of 30% sodium thiosulfate pentahydrate solution. Then, 750 ml tert-butyl methyl ether were added and the organic layer was separated. The aqueous layer was extracted three times with 750-ml portions of tert-butyl methyl ether and the combined organic layers were dried (sodium sulfate) and evaporated. Flash chromatography gave 68.9 g (63 %) of the title compound as light brown crystals.

MS m/e (%): 406 (M+H^{+,} 100).

d) 2,2-Dimethyl-N-(6-thiomorpholin-4-yl-4-o-tolyl-pyridin-3-yl)-propionamide

A mixture of 4.05 g (10.0 mmol) N-(4-iodo-6-thiomorpholin-4-yl-pyridin-3-yl)-2,2-dimethyl-propionamide, 54 ml toluene, 16 ml 2 N sodium carbonate solution, 347 mg (0.3 mmol) tetrakis(triphenylphosphine)palladium(0), 67 mg (0.3 mmol) palladium(II) acetate and 1.50 g (11.0 mmol) o-tolylboronic acid was heated under argon at 80° C. for 18 h. After cooling to room temperature, the aqueous phase was separated and washed twice with ethyl acetate. The combined organic layers were washed with 50 ml brine, dried (sodium sulfate) and evaporated. Purification by flash-chromatography gave 3.57 g (quantitative) of the title compound as a light brown solid.

MS m/e (%): 392 (M+Na^{+,} 4), 370 (M+H^{+,} 100).

e) 6-Thiomorpholin-4-yl-4-o-tolyl-pyridin-3-ylamine

A suspension of 3.45 g (9.3 mmol) 2,2-dimethyl-N-(6-thiomorpholin-4-yl-4-o-tolyl-pyridin-3-yl)-propionamide in 95 ml 3 N hydrochloric acid solution was heated under argon at 110° C. overnight. The reaction mixture was cooled to room temperature, washed with two 100-ml portions of diethyl ether and filtered over celite. The filtrate was diluted with 20 ml water and was adjusted to pH 11 by addition of 28 % sodium hydroxide solution under ice cooling. The product was extracted with three 100-ml portions of dichloromethane. The combined organic layers were washed with 50 ml brine, dried (sodium sulfate) and evaporated to give 2.53 g (95 %) of the title compound as a brown solid.

MS m/e (%): 286 (M+H^{+,} 100).

f) Methyl-(6-thiomorpholin-4-yl-4-o-tolyl-pyridin-3-yl)-amine

To a solution of 2.46 g (8.6 mmol) 6-thiomorpholin-4-yl-4-o-tolyl-pyridin-3-ylamine in 38 ml tetrahydrofuran were added 2.38 g (17 mmol) potassium carbonate (dissolved in 25 ml water) and 1.03 g (9.5 mmol) ethyl cloroformate. The reaction mixture was stirred for 1 h at room temperature and evaporated to remove tetrahydrofuran. The aqueous layer was extracted twice with 50-ml portions of dichloromethane and the organic layer was dried (sodium sulfate) and evaporated in vacuo. The residual oil was dissolved in 30 ml tetrahydrofuran and 7.4 ml (2.6 mmol) 3.5 M sodium bis(2-methoxyethoxy)aluminum hydride solution in toluene were added within 30 min. The reaction mixture was stirred at 50° C. overnight. After cooling to 0° C., 7.5 ml 1 N sodium hydroxide solution were added dropwise. Tetrahydrofuran was removed in vacuo and 10 ml of water were added. The aqueous layer was extracted twice with 20-ml portions of dichloromethane and the combined organic layers were dried (sodium sulfate), evaporated and purified by flash chromatography to give 2.37 g (92 %) of the title compound as a yellow solid.

MS m/e (%): 300 (M+H^{+,} 100).

g) 2-(3,5-Bis-trifluoromethyl-phenyl)-N-methyl-N-(6-thiomorpholin-4-yl-4-o-tolyl-pyridin-3-yl) -isobutyramide

A solution of 2.32 g (7.7 mmol) methyl-(6-thiomorpholin-4-yl-4-o-tolyl-pyridin-3-yl) -amine and 1.50 g (11.6 mmol) N-ethyldiisopropylamine in 20 ml tetrahydrofuran was cooled in an ice bath and 2.72 g (8.5 mmol) 2-(3,5-bis-trifluoromethyl-phenyl)-2-methyl-propionyl chloride were added dropwise. The reaction mixture was stirred at room temperature overnight and evaporated in vacuo. The residue was suspended in 200 ml 1 N sodium carbonate solution and extracted three times with 200-ml portions of ethyl acetate. The combined organic layers were dried (sodium sulfate) and evaporated. The residue was crystallized from ethanol to give 3.60 g (80 %) of the title compound as white crystals.

MS m/e (%): 582 (M+H^{+,} 100).

h) 2-(3,5-Bis-trifluoromethyl-phenyl)-N-[6-(1,1-dioxo-1λ⁶-thiomorpholin-4-yl)-4-o-tolyl-pyridin-3-yl]-N-methyl-isobutyramide

To a solution of 1.00 g (1.72 mmol) 2-(3,5-bis-trifluoromethyl-phenyl)-N-methyl-N-(6-thiomorpholin-4-yl-4-o-tolyl-pyridin-3-yl)-isobutyramide in 10 ml methanol were added 1.59 g (2.58 mmol) OXONE®. After stirring for 2 days at room temperature, 5 ml 38 % sodium hydrogensulfite solution and 20 ml saturated sodium carbonate solution were added consecutively and methanol was removed in vacuo. The residue was diluted with 25 ml water and extracted with three 25-ml portions of dichloromethane. The combined organic layers were dried (sodium sulfate), purified by flash chromatography and crystallized from ethanol to give 980 mg (93 %) of the title compound as white crystals. M.p. 200-201° C.

MS m/e (%): 636 (M+Na^{t}, 20), 614 (M+H^{+,} 100).

### Preparation of 2-(3,5-Bis-trifluoromethyl-phenyl)-N-[6- (1,1-dioxo-1λ⁶-thiomorpholin-4-yl)-4-(4-fluoro-2-methyl-phenyl)-pyridin-3-yl]-N-methyl-isobutyramide

The title compound was obtained as white crystals in comparable yields according to the procedures described above for the preparation of 2-(3,5-bis-trifluoromethyl-phenyl)-N-[6-(1,1-dioxo-1λ⁶-thiomorpholin-4-yl) -4-o-tolyl-pyridin-3 -yl]-N-methyl-isobutyramide using 4-fluoro-2-methyl-phenylboronic acid instead of o-tolylboronic acid in step d). M.p. 162.1-163.6° C.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

1. A method of treating benign prostatic hyperplasia in a mammal by administering to the mammal an effective amount of an NK-1 receptor antagonist.

2. The method according to claim 1, wherein the NK-1 receptor antagonist is a compound of the general formula (I) 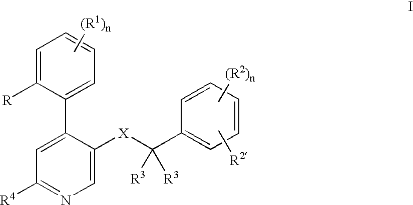
wherein
R is hydrogen, lower alkyl, lower alkoxy, halogen or trifluoromethyl;
R¹ is hydrogen or halogen; or
R and R¹ may be together -CH═CH-CH═CH-;
R² and R² are independently from each other hydrogen, halogen, trifluoromethyl, lower alkyl, lower alkoxy or cyano; or
R² and R²′may be together -CH═CH-CH═CH-, optionally substituted by one or two substituents selected from lower alkyl, halogen or lower alkoxy;
R³ is, independently from each other if occurring twice, hydrogen, lower alkyl or may, if occurring twice, form together with the carbon atom to which they are attached a cycloalkyl group;
R⁴ is hydrogen, -N(R⁵)₂, -N(R⁵) (CH₂)ₙOH, -N(R⁵)S(O)₂-lower alkyl, -N(R⁵)S(O)₂-phenyl, -N═CH-N(R⁵)₂, -N(R⁵)C(O)R⁵, a cyclic tertiary amine of the group or the group 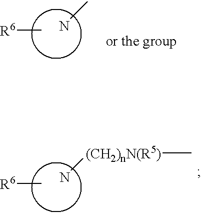
or R⁴ is -(C=-C)ₙR⁷ or -(CR′═CR″)ₙR⁷ wherein R⁷ is
a) halogen,
b) cyano, or the following groups:
c) -(CR′R″)ₙ-R⁸,
d) -C(O)NR′R″,
e) -C(O)O(CH₂)ₙR⁸,
f) -C(O)R⁸,
g) -N(OH)-(CH₂)ₙR⁸,
h) -NR′C(O)-(CH₂)ₙR⁸,
i) -N[C(O)-R′]₂,
j) -OR⁹,
k) -(CH₂)ₙ-SR⁹, -(CH₂)ₙ-S(O)R⁹, or -(CH₂)ₙ-S(O)₂R⁹,
l) aryl, optionally substituted by one or more substituents, selected from halogen, trifluoromethyl, lower alkyl, lower alkoxy, cyano, hydroxy, -NR′R″, nitro, -(CH₂)ₘOR′, -C(O)NR′R″, -C(O)OR′ or -C(O)R′,
m) is a five or six membered heteroaryl group, containing one to four heteroatoms, selected from N, O or S and may be optionally substituted by one or more substituents, selected from halogen, trifluoromethyl, lower alkyl, lower alkoxy, cyano, hydroxy, -NR′R″, nitro, -(CH₂)ₘOR′, -C(O)OR′, -C(O)NR′R″ or -C(O)R′,
n) is a five or six membered saturated cyclic tertiary amine of the group 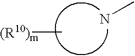
which may contain one additional heteroatom, selected from N, 0 or S, R′/R″ are independently from each other hydrogen, hydroxy, lower alkyl, cycloalkyl or aryl, wherein the lower alkyl, cycloalkyl or aryl group may be optionally substituted by one or more substituents, selected from halogen, trifluoromethyl, lower alkyl, lower alkoxy, cyano, hydroxy, -NR′″R″″, nitro, -(CH₂)ₘOR′″, -C(O)NR′″R″″, -C(O)OR′″ or -C(O)R′″,
R′″/R″″ are independently from each other hydrogen, lower alkyl, cycloalkyl or aryl, R⁸ is hydrogen, cyano, hydroxy, halogen, trifluoromethyl, -C(O)OR″, -OC(O)R or aryl, optionally substituted by one or more substituents, selected from halogen, trifluoromethyl, lower alkyl, lower alkoxy, cyano, hydroxy, -NR′R″, nitro, -(CH₂)ₘOR′, -C(O)NR′R″, -C(O)OR′ or -C(O)R″, or is a five or six membered heteroaryl group, containing one to four heteroatoms, selected from N, O or S and may be optionally substituted by one or more substituents, selected from halogen, trifluoromethyl, lower alkyl, lower alkoxy, cyano, hydroxy, -NR′R″, nitro, -(CH₂)ₘOR′, -C(O)NR′R″, -C(O)OR′ or-C(O)R′,
R⁹ is hydrogen, lower alkyl, trifluoromethyl, or aryl, wherein the lower alkyl or aryl group may be optionally substituted by one or more substituents, selected from halogen, trifluoromethyl, lower alkyl, lower alkoxy, cyano, hydroxy, -NR′R″, nitro, -C(O)NR′R″, -(CH₂)ₘOR′, -C(O)OR′ or -C(O)R′, or is a five or six membered heteroaryl group, containing one to four heteroatoms, selected from N, O or S and may be optionally substituted by one or more substituents, selected from halogen, trifluoromethyl, lower alkyl, lower alkoxy, cyano, hydroxy, -NR′R″, nitro, -(CH₂)ₘOR′, -C(O)NR′R″, -C(O)OR′ or-C(O)R′,
R¹⁰ is -C(O)-(CH₂)ₙOH or an oxo group; or R⁴ is an N-oxide of the general formula 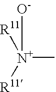
wherein R¹¹ and R^{11′}are independently from each other -(CH₂)ₚOR¹² or lower alkyl, wherein R¹² is hydrogen, lower alkyl or phenyl; or
R¹¹ and R^{11′}form together with the N-atom to which they are attached a cyclic tertiary amine of the group 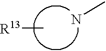
wherein R¹³ is hydrogen, hydroxy, lower alkyl, lower alkoxy, -(CH₂)ₚOH, -COOR³, -CON(R³)₂, -N(R³)CO-lower alkyl or -C(O)R³;
R⁵ is, independently from each other, hydrogen, C₃-₆-cycloalkyl, benzyl, phenyl or lower alkyl;
R⁶ is hydrogen, hydroxy, lower alkyl, -(CH₂)ₙCOO-lower alkyl, -N(R⁵)CO-lower alkyl, hydroxy-lower alkyl, cyano, -(CH₂)ₙO(CH₂)ₙOH, -CHO or a 5-or 6 membered heterocyclic group, optionally bonded via an alkylene group;
X is -C(O)N(R⁵)-, -(CH₂)ₚO-, -O(CH₂)ₚ-, -(CH₂)ₚN(R⁵)-, -N(R⁵)C(O)-, or -N(R⁵)(CH₂)ₚ-;
n is 0, 1, 2, 3 or 4;
m is 1 or2; and
p is 1, 2 or 3;
and the pharmaceutically acceptable acid addition salts and the prodrugs thereof.

3. The method according to claim 1, wherein the NK-1 receptor antagonist is a compound of general formula (I) 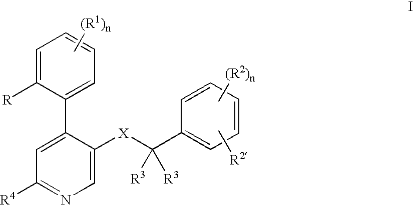
wherein
R is hydrogen, lower alkyl, lower alkoxy, halogen or trifluoromethyl;
R¹ is hydrogen or halogen; or
R and R¹ may be together -CH═CH-CH═CH-;
R² and R^{2′}are independently from each other hydrogen, halogen, trifluoromethyl, lower alkoxy or cyano; or
R² and R^{2′}may be together -CH═CH-CH═-CH-, optionally substituted by one or two substituents selected from lower alkyl or lower alkoxy;
R³ is hydrogen, lower alkyl or form a cycloalkyl group;
R⁴ is hydrogen, -N(R⁵)₂, -N(R⁵)(CH₂)ₙOH, -N(R⁵)S(O)₂-lower alkyl, -N(R ⁵)S(O)₂-phenyl,-N═-CH-N(R⁵)₂, -N(R⁵)C(O)R⁵ or a cyclic tertiary amine of the group 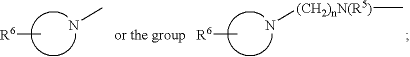
R⁵ is, independently from each other, hydrogen, C₃-₆-cycloalkyl, benzyl or lower alkyl;
R⁶ is hydrogen, hydroxy, lower alkyl, -(CH₂)ₙCOO-lower alkyl, -N(R⁵)CO-lower alkyl, hydroxy-lower alkyl, cyano, -(CH₂)ₙO(CH₂)ₙOH, -CHO or a 5-or 6 membered heterocyclic group, optionally bonded via an alkylene group;
X is -C(O)N(R⁵)-,-(CH₂)ₘO-, -(CH₂)ₘN(R⁵)-, -N(R⁵)C(O)-, or -N(R⁵)(CH₂)ₘ-;
n is0, 1, 2, 3or 4;and
m is 1or 2;
and the pharmaceutically acceptable acid addition salts and the prodrugs thereof.

4. The method according to claim 2 or claim 3, wherein the NK-1 receptor antagonist is a compound of general formula (I), wherein X is -C(O)N(R ⁵) - and R⁵ is methyl, ethyl or cyclopropyl.

5. The method according to claim 4, wherein the compound is selected from the group consisting of
N-(3,5-bis-trifluoromethyl-benzyl)-N-methyl-4-o-tolyl-nicotinamide,
N-(3,5-bis-trifluoromethyl-benzyl)-N-methyl-4-(2-chloro-phenyl)-nicotinamide,
N-(3,5-bis-trifluoromethyl-benzyl)-N-methyl-4-(2-trifluoromethyl-phenyl)-nicotinamide,
N-(3 ,5-bis-trifluoromethyl-benzyl)-N-methyl-4-(2-fluoro-phenyl)-nicotinamide,
N-(3,5-bis-trifluoromethyl-benzyl)-N-methyl-4-(2-methoxy-phenyl)-nicotinamide,
N-(3,5-bis-trifluoromethyl-benzyl)-N-methyl-4-phenyl-nicotinamide,
N-(3,5-bis-trifluoromethyl-benzyl)-N-ethyl-4-o-tolyl-nicotinamide,
N-(3,5-bis-trifluoromethyl-benzyl) -N-cyclopropyl-4-o-tolyl-nicotinamide,
N-[1-(3,5-bis-trifluoromethyl-phenyl)-ethyl]-N-methyl-4-o-tolyl-nicotinamide,
N-(3,5-di-fluorobenzyl) -N-methyl-4-o-tolyl-nicotinamide,
N-(3,5-di-chlorobenzyl)-N-methyl-4-o-tolyl-nicotinamide,
N-(3,5-bis-trifluoromethyl-benzyl)-N-methyl-6-(4-methyl-piperazin-1-yl)-4-o-tolyl-nicotinamide,
2′-methyl-5-(4-methyl-piperazin-1-yl)-biphenyl-2-carboxylic acid-(3,5-bis-trifluoromethyl-benzyl) -methyl-amide,
N-(3,5-bis-trifluoromethyl-benzyl)-N-methyl-6-(4-methyl-piperazin-1-yl)-4-naphthalen-1-yl-nicotinamide,
(4-{5- [(3,5-bis-trifluoromethyl-benzyl) -methyl-carbamoyl]-4-o-tolyl-pyridin-2-yl}-piperazin-1-yl)-acetic acid ethyl ester,
5′- [(3,5-bis-trifluoromethyl-benzyl) -methyl-carbamoyl]-4′-o-tolyl-3,4,5,6-tetrahydro-2H- [1,2′]bipyridinyl-4-carboxylic acid ethyl ester,
N-(3,5-b is-trifluoromethyl-benzyl) -N-methyl-6-(4-propyl-piperazin-1-yl) -4-o-tolyl-nicotinamide,
(RS) -6- [3- (acetyl-methyl-amino) -pyrrolidin-1-yl]-N- (3,5-bis-trifluoromethyl-benzyl) -N-methyl-4-o-tolyl-nicotinamide,
N-(3,5-bis-trifluoromethyl-benzyl)-N-methyl-6-[methyl-(2-morpholin-4-yl-ethyl)-amino]-4-o-tolyl-nicotinamide,
N-(3,5-bis-trifluoromethyl-benzyl)-N-methyl-6-morpholin-4-yl-4-o-tolyl-nicotinamide,
N-(3,5-bis-trifluoromethyl-benzyl) -N-methyl-6-thiomorpholin-4-yl-4-o-tolyl-nicotinamide,
N-(3,5-bis-trifluoromethyl-benzyl)-N-methyl-6-(1-oxo-1λ⁴-thiomorpholin-4-yl)-4-o-tolyl-nicotinamide,
N-(3,5-bis-trifluoromethyl-benzyl)-6-(1,1-dioxo-1λ6-thiomorpholin-4-yl)-N-methyl-4-o-tolyl-nicotinamide,
N-(3,5-bis-trifluoromethyl-benzyl)-N-methyl-6-piperazin-1-yl-4-o-tolyl-nicotinamide,
N-(3,5-bis-trifluoromethyl-benzyl)-6- [4-(2-hydroxy-ethyl)-piperazin-1-yl]-N-methyl-4-o-tolyl-nicotinamide,
N-(3,5-bis-trifluoromethyl-benzyl)-6-(4-cyanomethyl-piperazin-1-yl)-N-methyl-4-o-tolyl-nicotinamide,
N-(3,5-bis-trifluoromethyl-benzyl)-6-{4-[2- (2-hydroxy-ethoxy)-ethyl]-piperazin-1-yl]-N-methyl-4-o-tolyl-nicotinamide,
N-(3,5-bis-trifluoromethyl-benzyl)-N-methyl-6-(4-[1,2,4]oxadiazol-3- yl-methyl-piperazin-1-yl)-4-o-tolyl-nicotinamide,
N-(3,5-bis-trifluoromethyl-benzyl)-N-methyl-6-[4-(5-oxo-4,5-dihydro-1H-[1,2,4]triazol-3-yl-methyl) -piperazin-1-yl]-4-o-tolyl-nicotinamide,
N-(3,5-bis-trifluoromethyl-benzyl)-6-(4-formyl-piperazin-1-yl)-N-methyl-4-o-tolyl-nicotinamide, and
N-methyl-N-(2-methyl-naphthalen-1-yl-methyl)-6-morpholin-4-yl-4-o-tolyl-nicotinamide; or a pharmaceutically acceptable acid addition salt thereof.

6. The method according to claim 2 or claim 3, wherein the NK-1 receptor antagonist is a compound of general formula (I), wherein X is -N(R⁵)-C(O)- and R⁵ is hydrogen or methyl.

7. The method according to claim 6, wherein the compound is selected from the group consisting of
2-(3,5-bis-trifluoromethyl-phenyl)-N-methyl-N-[6-(4-methyl-piperazin-1-yl)-4-o-tolyl-pyridin-3-yl]-isobutyramide,
2-(3,5-bis-trifluoromethyl-phenyl)-N-[4-(2-chloro-phenyl)-6-(4-methyl-piperazin-1-yl) -pyridin-3-yl]-N-methyl-isobutyramide,
2-(3,5-bis-trifluoromethyl-phenyl)-N-[4-(4-fluoro-2-methyl-phenyl)-6-(4-methyl-piperazin-1-yl) -pyridin-3-yl]-N-methyl-isobutyramide,
2-(3,5-bis-trifluoromethyl-phenyl)-N-[4-(2-chloro-phenyl)-pyridin-3-yl]-N-methyl-isobutyramide,
2-(3,5-bis-trifluoromethyl-phenyl-N-methyl-N-(4-o-tolyl-pyridin-3-yl)-isobutyramide,
2-(3,5-bis-trifluoromethyl-phenyl)-N-(4-o-tolyl-pyridin-3-yl)-isobutyramide,
2-(3,5-bis-trifluoromethyl-phenyl)-N-methyl-N-(4-o-tolyl-pyridin-3-yl)-acetamide,
2-(3,5-bis-trifluoromethyl-phenyl)-N-methyl-N-(4-o-tolyl-pyridin-3-yl)-propionamide,
2-(3,5-bis-trifluoromethyl-phenyl)-N-methyl-N-(6-morpholin-4-yl-4-o-tolyl-pyridin-3-yl) -isobutyramide,
2-(3,5-bis-trifluoromethyl-phenyl)-N-[4-(2-chloro-phenyl) -6-morpholin-4-yl-pyridin-3-yl]-N-methyl-isobutyramide,
2-(3,5-bis-trifluoromethyl-phenyl)-N-methyl-N-{6-[methyl-(2-morpholin-4-yl-ethyl) -amino]-4-o-tolyl-pyridin-3-yl}-isobutyramide,
2-(3,5-bis-trifluoromethyl-phenyl)-N-methyl-N-[6-(4-pyrimidin-2-yl-piperazin-1-yl)-4-o-tolyl-pyridin-3-yl]-isobutyramide,
2-(3,5-bis-trifluoromethyl-phenyl)-N-(6-morpholin-4-yl-4-o-tolyl-pyridin-3-yl)-isobutyramide,
2-(3,5-bis-trifluoromethyl-phenyl)-N-[4-(2-chloro-phenyl)-6-dimethylamino-pyridin-3-yl]-isobutyramide,
2-(3,5-bis-trifluoromethyl-phenyl)-N-methyl-N-(6-piperazin-1-yl-4-o-tolyl-pyridin-3-yl)-isobutyramide,
2-(3,5-bis-trifluoromethyl-phenyl)-N-(4-hydroxy-4′-o-tolyl-3,4,5,6-tetrahydro-2H-1,2′] bipyridinyl-5′-yl) -N-methyl-isobutyramide,
2-(3,5-bis-trifluoromethyl-phenyl)-N-{6- (2-hydroxy-ethyl) -methyl-amino]-4-o-tolyl-pyridin-3-yl}-N-methyl-isobutyramide,
(R)-2-(3,5-bis-trifluoromethyl-phenyl)-N-[6-(3-hydroxy-pyrrolidin-1-yl) -4-o-tolyl-pyridin-3-yl]-N-methyl-isobutyramide,
2-(3,5-bis-trifluoromethyl-phenyl)-N-methyl-N-(6-morpholin-4-yl-4-o-tolyl-pyridin-3-yl)-acetamide, and
[2-(3,5-bis-trifluoromethyl-phenyl)-2-methyl-propyl]-[4-(4-fluoro-2-methyl-phenyl) -6- (4-methyl-piperazin-1-yl) -pyridin-3-yl]-methylamine; or a pharmaceutically acceptable acid addition salt thereof

8. The method according to claim 4, wherein the compound is selected from the group consisting of
N-(3,5-bis-trifluoromethyl-benzyl)-N-methyl-4-o-tolyl-6-[1,2,4]triazol-1-yl-nicotinamide,
N-(3,5-bis-trifluoromethyl-benzyl)-6-(2-hydroxy-ethylamino)-N-methyl-4-o-tolyl-nicotinamide,
4-hydroxy-4′-o-tolyl-3,4,5,6-tetrahydro-2H-[1,2′]bipyridinyl-5′-carboxylic acid (3,5-bis-trifluoromethyl-benzyl)-methyl-amide,
4-(2-hydroxy-ethoxy) -4′-o-tolyl-3,4,5,6-tetrahydro-2H-[1,2′]bipyridinyl-5′-carboxylic acid (3,5-bis-trifluoromethyl-benzyl)-methyl-amide,
(R)-N-(3,5-bis-trifluoromethyl-benzyl)-6-(3-hydroxy-pyrrolidin-1-yl)-N-methyl-4-o-tolyl-nicotinamide, and
4′-(2-chloro-phenyl)-4-hydroxy-3,4,5,6-tetrahydro-2H-[1,2′]bipyridinyl-5′-carboxylic acid (3,5-bis-trifluoromethyl-benzyl)-methyl-amide; or a pharmaceutically acceptable acid addition salt thereof

9. The method according to claim 6, wherein the compound is selected from the group consisting of
2-(3,5-bis-trifluoromethyl-phenyl)-N-[6-(2-hydroxy-ethylamino)-4-o-tolyl-pyridin-3-yl]-N-methyl-isobutyramide,
2-(3,5-bis-trifluoromethyl-phenyl)-N-[6-(2,3-dihydro- [1,4]oxazin-4-yl)-4-o-tolyl-pyridin-3-yl]-N-methyl-isobutyramide,
N-(6-acetylamino-4-o-tolyl-pyridin-3-yl)-2-(3,5-bis-trifluoromethyl-phenyl)-N-methyl-isobutyramide,
N-[6-(acetyl-methyl-amino)-4-o-tolyl-pyridin-3-yl]-2-(3,5-bis-trifluoromethyl-phenyl)-N-methyl-isobutyramide,
cyclopropanecarboxylic acid (5-{[2-(3,5-bis-trifluoromethyl-phenyl)-2-methyl-propionyl]-methyl-amino}-4-o-tolyl-pyridin-2-yl) -amide,
cyclopropanecarboxylic acid (5-{[2-(3,5-bis-trifluoromethyl-phenyl)-2-methyl-propionyl]-methyl-amino}-4-o-tolyl-pyridin-2-yl)-methyl-amide,
2-(3,5-bis-trifluoromethyl-phenyl)-N-(6-imidazol-1-yl-4-o-tolyl-pyridin-3-yl)-N-methyl-isobutyramide, and
2-(3,5-bis-trifluoromethyl-phenyl)-N-[4-(2-chloro-phenyl)-6- (2-hydroxy-ethylamino)-pyridin-3-yl]-N-methyl-isobutyramide; or a pharmaceutically acceptable acid addition salt thereof

10. The method according to claim 2 or 3, wherein the NK-1 receptor antagonist is a compound of general formula (I), wherein R⁴ is -(C≡C)ₙR⁷ or -(CR′═CR″)ₙR⁷.

11. The method according to claim 10, wherein the NK-1 receptor antagonist is a compound according to formula (I), wherein in R⁴ is -(C≡C)ₙR⁷ or -(CR′═CR″)ₙR⁷ and X is -C(O)N(CH₃)- and (R²)n is 3,5-di-CF₃.

12. The method according to claim 11, wherein the compound is selected from the group consisting of
N-(3,5-bis-trifluoromethyl-benzyl)-6-(4-hydroxyacetyl-piperazin-1-yl)-N-methyl-4-o-tolyl-nicotinamide,
N-(3,5-bis-trifluoromethyl-benzyl)-6-chloro-N-methyl-4-o-tolyl-nicotinamide,
N-(3,5-bis-trifluoromethyl-benzyl)-6-cyanomethyl-N-methyl-4-o-tolyl- nicotinamide,
N-(3,5-bis-trifluoromethyl-benzyl)-6-iodo-N-methyl-4-o-tolyl-nicotinamide,
4-o-tolyl-[2,4′]bipyridinyl-5-carboxylic acid (3,5-bis-trifluoromethyl-benzyl)-methyl-amide,
5- [(3,5-bis-trifluoromethyl-benzyl)-methyl-carbamoyl]-4-o-tolyl-pyridine-2-carboxylic acid methyl ester,
N-(3,5-bis-trifluoromethyl-benzyl)-6-hydroxymethyl-N-methyl-4-o-tolyl-nicotinamide,
6-(5-acetyl-thiophen-2-yl)-N-(3,5-bis-trifluoromethyl-benzyl)-N-methyl-4-o-tolyl-nicotinamide,
4-o-tolyl-1′,2′,3′,6′-tetrahydro-[2,4′]bipyridinyl-5-carboxylic acid (3,5-bis-trifluoromethyl-benzyl)-methyl-amide,
N-(3,5-bis-trifluoromethyl-benzyl)-6-(4-hydroxymethyl-phenyl)-N-methyl-4-o-tolyl-nicotinamide,
2′-methyl-4-o-tolyl-[2,4′]bipyridinyl-5-carboxylic acid (3,5-bis-trifluoromethyl-benzyl)-methyl-amide,
N-(3,5-bis-trifluoromethyl-b enzyl) -N-methyl-6-(3-methyl-[1,2,4]oxadiazol-5-yl) -4-o-tolyl-nicotinamide,
6-(3-amino-prop- I -ynyl) -N- (3,5-bis-trifluoromethyl-benzyl) -N-methyl-4-o-tolyl-nicotinamide,
-(RS)-N-(3,5-bis-trifluoromethyl-benzyl)-6-(2-hydroxy-ethanesulfinylmethyl)-N-methyl-4-o-tolyl-nicotinamide,
N-(3,5-bis-trifluoromethyl-benzyl) -N-methyl-6-(1 -methyl-1H-imidazol-2-yl-sulfanylmethyl) -4-o-tolyl-nicotinamide,
(RS)-N-(3,5-bis-trifluoromethyl-benzyl)-N-methyl-6-(pyridine-2-sulfinyl)-4-o-tolyl-nicotinamide,
N-(3,5-bis-trifluoromethyl-benzyl)-N-methyl-6-(pyridine-2-sulfonyl)-4-o-tolyl-nicotinamide, and
N-(3,5-bis-trifluoromethyl-benzyl)-6-(3-hydroxy-propoxy)-N-methyl-4-o-tolyl-nicotinamide; or a pharmaceutically acceptable acid addition salt thereof.

13. The method according to claim 10, wherein the NK-1 receptor antagonist is a compound of general formula (I), wherein R⁴ is -(C=-C)ₙR⁷ or -(CR′═CR″)ₙR⁷ and X is -N(CH₃)C(O)- and (R²)ₙ is 3,5-di-CF₃.

14. The method according to claim 13, wherein the compound is selected from the group consisting of
2-(3,5-bis-trifluoromethyl-phenyl)-N-{6-[hydroxy-(2-hydroxy-ethyl)-amino]-4-o-tolyl-pyridin-3-yl}-N-methyl-isobutyramide,
2-(3,5-bis-trifluoromethyl-phenyl)-N-methyl-N-[6-(3-oxo-morpholin-4-yl)-4-o-tolyl-pyridin-3-yl]-isobutyramide,
acetic acid (5-{[2-(3,5-bis-trifluoromethyl-phenyl)-2-methyl-propionyl]-methyl-amino}-4-o-tolyl-pyridin-2-ylcarbamoyl)-methyl ester,
2-(3,5-bis-trifluoromethyl-phenyl)-N-[6-(2-hydroxy-acetylamino)-4-o-tolyl-pyridin-3-yl]-N-methyl-isobutyramide,
2-(3,5-bis-trifluoromethyl-phenyl)-N-[6-(hydroxyacetyl-methyl-amino)-4-o-tolyl-pyridin-3-yl]-N-methyl-isobutyramide,
2-(3,5-bis-trifluoromethyl-phenyl) -N-[6-(2,5-dioxo-pyrrolidin-1-yl) -4-o-tolyl-pyridin-3-yl]-N-methyl-isobutyramide,
cyclopropanecarboxylic acid (5-{[2-(3,5-bis-trifluoromethyl-phenyl)-2-methyl-propionyl]-methyl-amino}-4-o-tolyl-pyridin-2-yl)-cyclopropanecarbonyl-amide,
2-(3,5-bis-trifluoromethyl-phenyl)-N-(6-chloro-4-o-tolyl-pyridin-3-yl)-N-methyl-isobutyramide,
2-(3,5-bis-trifluoromethyl-phenyl)-N-[4-(2-chloro-phenyl)-2′-methyl-[2,4′]bipyridinyl-5-yl]-N-methyl-isobutyramide,
2-(3,5-bis-trifluoromethyl-phenyl)-N-(6-ethynyl-4-o-tolyl-pyridin-3-yl)-N-methyl-isobutyramide,
2-(3,5-bis-trifluoromethyl-phenyl)-N-[6-(3-hydroxymethyl-isoxazol-5-yl)-4-o-tolyl-pyridin-3-yl]-N-methyl-isobutyramide,
2-(3,5-bis-trifluoromethyl-phenyl)-N-[6-(3-hydroxy-prop-1-ynyl)-4-o-tolyl-pyridin-3-yl]-N-methyl-isobutyramide, and
(RS)-2-(3,5-bis-trifluoromethyl-phenyl)-N- [6-(3-methoxy-benzenesulfinyl)-4-o-tolyl-pyridin-3-yl]-N-methyl-isobutyramide; or a pharmaceutically acceptable acid addition salt thereof.

15. The method according to claim 10, wherein the NK-1 receptor antagonist a compound of general formula (I), wherein R⁴ is -(C≡C)ₙR^{11′}or -(CR′═CR″)ₙR^{11′}and R³ and R^{3′}are both methyl and R is chloro.

16. The method according to claim 15, wherein the compound is 2-(3,5-bis-trifluoromethyl-phenyl)-N-{4-(2-chloro-phenyl)-6-[hydroxy-(2-hydroxy-ethyl) -amino]-pyridin-3-yl}-N-methyl-isobutyramide or is 2-(3,5-bis-trifluoromethyl-phenyl)-N-[4-(2-chloro-phenyl)-6-(3-oxo-morpholin-4-yl) -pyridin-3-yl]-N-methyl-isobutyramide or is a pharmaceutically acceptable acid addition salt thereof

17. The method according to claim 2 or 3, wherein the NK-1 receptor antagonist is a compound of general formula (I), wherein R⁴ is an N-oxide of the general formula 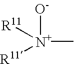
and X is -C(O)N(R⁵)- and R⁵ is methyl or X is -N(R⁵)-C(O)- and R⁵ is hydrogen or methyl.

18. The method according to claim 17, wherein the compound is selected from the group consisting of
4-{5-[(3,5-bis-trifluoromethyl-benzyl)-methyl-carbamoyl]-4-o-tolyl-pyridin-2-yl}-4-oxy-piperazine-1-carboxylic acid tert-butyl ester,
5′-[(3,5-bis-trifluoromethyl-benzyl) -methyl-carbamoyl]-4′-o-tolyl-1-oxy-3,4,5,6-tetrahydro-2H-[1,2′]bipyridinyl-4-carboxylic acid ethyl ester,
(RS)-6-[3-(acetyl-methyl-amino)-1-oxo-pyrrolidin-1-yl]-N-(3,5-bis-trifluoromethyl-benzyl)-N-methyl-4-o-tolyl-nicotinamide,
N-(3,5-bis-trifluoromethyl-benzyl)-N-methyl-6-(4-oxy-morpholin-4-yl)-4-o-tolyl-nicotinamide,
N-(3,5-bis-trifluoromethyl-benzyl)-6-(1,1-dioxo-1X⁶-4-oxy-thiomorpholin-4-yl)-N-methyl-4-o-tolyl-nicotinamide,
N-(3,5-bis-trifluoromethyl-benzyl)-6-(4-formyl-1-oxy-piperazin-1-yl)-N-methyl-4-o-tolyl-nicotinamide,
N-methyl-N-(2-methyl-naphthalen-1-yl-methyl)-6-(4-oxy-morpholin-4-yl)-4-o-tolyl-nicotinamide,
N-methyl-6-(4-oxy-morpholin-4-yl) -N-naphthalen-1-yl-methyl-4-o-tolyl-nicotinamide,
N-(2-methoxy-naphthalen-1-yl-methyl)-N-methyl-6-(4-oxy-morpholin-4-yl)-4-o-tolyl-nicotinamide,
N-(2-methoxy-benzyl)-N-methyl-6-(4-oxy-morpholin-4-yl)-4-o-tolyl-nicotinamide,
N-(5-chloro-2-methoxy-benzyl)-N-methyl-6-(4-oxy-morpholin-4-yl)-4-o-tolyl-nicotinamide,
N-(2-chloro-5-methoxy-benzyl)-N-methyl-6-morpholin-4-yl-4-o-tolyl-nicotinamide,
N-methyl-6-(4-oxy-morpholin-4-yl)-N-pentafluorophenylmethyl-4-o-tolyl-nicotinamide,
N-methyl-6-(4-oxy-morpholin-4-yl)-N-naphthalen-2-yl-methyl-4-o-tolyl-nicotinamide,
N- [2-methoxy-5-(5-trifluoromethyl-tetrazol-1-yl)-benzyl]-N-methyl-6-(4-oxy-morpholin-4-yl) -4-o-tolyl-nicotinamide,
N-(1,4-dimethoxy-naphthalen-2-yl-methyl)-N-methyl-6-(4-oxy-morpholin-4-yl)-4-o-tolyl-nicotinamide,
5′-[(3,5-bis-trifluoromethyl-benzyl)-methyl-carbamoyl]-4′-o-tolyl-1-oxy-3,4,5,6-tetrahydro-2H-[1,2′]bipyridinyl-4-carboxylic acid,
2-(3,5-bis-trifluoromethyl-phenyl)-N-methyl-N-[6-(4-oxy-morpholin-4-yl)-4-o-tolyl-pyridin-3-yl]-isobutyramide,
2-(3,5-bis-trifluoromethyl-phenyl)-N-[4-(2-chloro-phenyl)-6-(4-oxy-morpholin-4-yl)-pyridin-3-yl]-N-methyl-isobutyramide,
2-(3,5-bis-trifluoromethyl-phenyl)-N-[6-(4-oxy-morpholin-4-yl)-4-o-tolyl-pyridin-3-yl]-isobutyramide,
2-(3,5-bis-trifluoromethyl-phenyl)-N-[4′-(2-chloro-phenyl)-1-oxy-3,4,5,6-tetrahydro-2H-[1,2′]bipyridinyl-5′-yl]-N-methyl-isobutyramide,
2-(3,5-bis-trifluoromethyl-phenyl)-N-(6-oxy-dimethylamino-4-o-tolyl-pyridin-3-yl)-N-methyl-isobutyramide,
2-(3,5-bis-trifluoromethyl-phenyl)-N-[4-(2-chloro-phenyl)-6-oxy-dimethylamino-pyridin-3-yl]-isobutyramide,
2-(3,5-bis-trifluoromethyl-phenyl)-N-i-(4-hydroxy-1-oxy-4′-o-tolyl-3,4,5,6-tetrahydro-2H-[1,2′]bipyridinyl-5′-yl)-N-methyl-isobutyramide,
2-(3,5-bis-trifluoromethyl-phenyl)-N-{6- [(2-hydroxy-ethyl)-1-oxy-methyl-amino]-4-o-tolyl-pyridin-3-yl}-N-methyl-isobutyramide,
(R)-2-(3,5-bis-trifluoromethyl-phenyl)-N-[6-(3-hydroxy-1-oxy-pyrrolidin-1-yl)-4-o-tolyl-pyridin-3-yl]-N-methyl-isobutyramide,
2-(3,5-bis-trifluoromethyl-phenyl)-N-methyl-N-[6-(4-oxy-morpholin-4-yl)-4-o-tolyl-pyridin-3-yl]-acetamide,
2-(3,5-dimethoxy-phenyl)-N-methyl-N-[6-(4-oxy-morpholin-4-yl)-4-o-tolyl-pyridin-3-yl]-acetamide, and
2-(3-fluoro-5-trifluoromethyl-phenyl)-N-methyl-N-[6-(4-oxy-morpholin-4-yl)-4-o-tolyl-pyridin-3-yl]-acetamide; or a pharmaceutically acceptable acid addition salt thereof.

19. The method according to claim 2 or 3, wherein the NK-1 receptor antagonist is 2-(3,5-bis-trifluoromethyl-phenyl)-N-methyl-N-(6-morpholin-4-yl-4-o-tolyl-pyridin-3-yl)-isobutyramide or is 2-(3,5-bis-trifluoromethyl-phenyl)-N-methyl-N-[6-(4-oxy-morpholin-4-yl)-4-o-tolyl-pyridin-3-yl]-isobutyramide or is a pharmaceutically acceptable acid addition salt thereof

20. The method according to claim 2 or 3, wherein the NK-1 receptor antagonist is 2-(3,5-bis-trifluoromethyl-phenyl)-N-methyl-N-[6-(4-methyl-piperazin-1-yl) -4-o-tolyl-pyridin-3-yl]-isobutyramide, 2-(3,5-bis-trifluoromethyl-phenyl)-N- [6-(1,1-dioxo-1λ⁶-thiomorpholin-4-yl)-4-o-tolyl-pyridin-3-yl]-N-methyl-isobutyramide, or 2-(3,5-bis-trifluoromethyl-phenyl)-N-[6-(1,1-dioxo-1λ⁶-thiomorpholin-4-yl)-4-(4-fluoro-2-methyl-phenyl)-pyridin-3-yl]-N-methyl-isobutyramide or pharmaceutically acceptable acid addition salts thereof.

21. The method according to claim 1, wherein the NK-1 receptor antagonist is a compound selected from the group of NK-1 receptor antagonists under drug development designated GR205171, HSP-117, L 703,606, L 668,169, LY 303241, LY 306740, MK-869, R-544, Spantide III, WIN-62,577, GR 103,537, L 758,298, NKP608, CGP49823, CP-96,345, CP-99,994, CP-122,721, FK 888, GR203040, GR 82334, GR 94800, L 732,138, L 733,060, L 742,694, L 754,030, LY 303870, MEN 11149, PD 154075, RP-67580, RPR 100893, Spendide, Spantide II, SR140333, WIN-41,708, WIN-62,577, SR-48,968, L-659,877, MEN 10627, SR 144190, GR 94800, SR-142,801, R820, R486, SB 222200, L 758,298, NK-608, CGP 47899 and MEN 11467; or is a pharmaceutically acceptable acid addition salt thereof.

22. The method of claim 1, wherein the NK-1 receptor antagonist has a pKi of greater than 7.

23. The method of claim 22, wherein the NK-1 receptor antagonist has a pKi of between 8 and 10.

24. The method of claims 1-3 and 22-23, wherein the mammal is a human.

25. A pharmaceutical composition comprising one or more NK-1 receptor antagonists as defined in claim 21 and a pharmaceutically acceptable excipient for the treatment of benign prostatic hyperplasia.
